(19) United States
(12) Patent Application Publication
(10) Pub. No.: US 2024/0197701 A1
(43) Pub. Date: Jun. 20, 2024

(54) ANDROGEN RECEPTOR INHIBITION TO TREAT SEPSIS AND SHOCK

(71) Applicants: RHODE ISLAND HOSPITAL, Providence, RI (US); APPLIED BIOLOGY, INC., Irvine, CA (US)

(72) Inventors: Gerard J. Nau, Sharon, MA (US); Carlos G. Wambier, West Greenwich, RI (US); Andy Goren, Newport Beach, CA (US)

(21) Appl. No.: 18/286,985

(22) PCT Filed: Apr. 19, 2022
(86) PCT No.: PCT/US2022/025387

§ 371 Date: Oct. 13, 2023

Related U.S. Application Data
(60) Provisional application No. 63/176,488, filed on Apr. 19, 2021.

(51) Int. Cl. A61K 31/4439; A61P 31/14

(52) U.S. Cl. CPC A61K 31/4439; A61P 31/14

(57) ABSTRACT

The invention provides a method for applying diarylhydantoin compounds, including flutamide, bicalutamide, enzalutamide, apalutamide, and proxalutamide, to treat sepsis and conditions that mimic sepsis.

### FIELD OF THE INVENTION

This invention generally relates to Infectious diseases, e.g., generalized sepsis.

### BACKGROUND OF THE INVENTION

Sepsis is a leading cause of death globally. According to the United States Centers for Disease Control, at least 1.7 million adults in America develop sepsis annually, nearly 270,000 people. One out of every three people who die in a hospital die of sepsis.

Animal studies and human observational studies have shown higher rates of sepsis and worse outcomes in males. Males also have worse outcomes in other conditions that mimic sepsis, such as hemorrhagic shock and trauma. More recently, similar gender disparities were seen with COVID-19, with men faring worse than women.

The mainstay of sepsis management is treating the underlying infection. The Surviving Sepsis Campaign evaluated several interventions without identifying treatments with a meaningful impact on survival.

There remains a need in the medical art for better medical management of sepsis.

### SUMMARY OF THE INVENTION

In one aspect, the invention provides that proxalutamide's beneficial effect is due to a general improvement in sepsis induced by SARS-COV-2.

In the first embodiment, the invention provides a method for applying diarylhydantoin compounds, including flutamide, bicalutamide, enzalutamide, apalutamide, and proxalutamide, to treat sepsis and conditions that mimic sepsis. The method can also include administering dutasteride or spironolactone to treat sepsis and conditions that mimic sepsis. This method is an improvement on previous methods of medical management because there are no medications besides antimicrobials for sepsis. The inventors administered proxalutamide given to COVID-19 patients. This standard of care dramatically reduced the severity of disease and mortality compared to placebo.

In the second embodiment, the invention can be used for the hundreds of thousands of Americans who suffer from traumatic injury.

In a third embodiment, the invention provides a method for applying 5 alpha-reductase inhibitors (dutasteride, finasteride) and spironolactone (another androgen receptor blocker). COVID-19 patients who started on dutasteride or spironolactone have more rapid symptom resolution and virus clearance (larger numbers on odds ratio are better in this analysis). The invention provides an “anti-androgen treatment” that is accomplished by androgen receptor blockers (diarylhydantoin compounds or spironolactone) or 5 alpha-reductase inhibitors.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 (TABLE 1) lists the baseline characteristics of the study populations.

FIG. 2 shows the survival estimate curves of the results of EXAMPLE 1.

FIG. 3 is a patient selection flow diagram.

FIG. 4 is a pair of Kaplan-Meier survivor function estimates. FIG. 4A shows the proportion of positive nasopharyngeal RT-PCR-SARS-COV-2. Tests were performed at 7-day intervals. Vertical lines in datapoints represent 95% confidence intervals, and the graph was simplified by smoothing in curves. FIG. 4B shows the proportion of symptomatic patients (symptoms excluded anosmia and ageusia).

FIG. 5 (TABLE 2) lists the baseline clinical characteristics, comorbidities, and concomitant medications of the studied population (IQR=interquartile range). The ordinal scale scores at baseline were 6, 5, and 4 in 66.5%, 30.4%, and 2.8% of the population, respectively. The distribution of ordinal scale score was similar between proxalutamide and placebo arms. Except for colchicine, all concomitant medications were used at similar proportions between the groups. Remdesivir was not a treatment option because of the emergency use authorization dated Mar. 12, 2021, in Brazil). See Brasil MDS. Anvisa aprova registro da vacina da Fiocruz/AstraZeneca e de medicamento contra o coronavírus— O imunizante e o antiviral Remdesivir podem ser usados amplamente no país. Published 2021. Compliance with the 14-day treatment was 91% and 89% for the proxalutamide and placebo arms, respectively. Because the study was conducted in Brazil, the population is ethnically mixed and most patients did not identify themselves as Caucasian, black, or Asian.

FIG. 6 is a decision chart showing the enrollment and randomization of the studied population. The median age was fifty years (interquartile range [IQR], forty-one years to 62), and forty-nine years (interquartile range, 38 to 61) for the proxalutamide and placebo groups, respectively. Patients with a body mass index (BMI) above 30 kg/m2, hypertension, type 2 diabetes mellitus (T2DM), and chronic obstructive pulmonary disorder (COPD) were equally distributed between study arms. No comorbidities, one comorbidity, and two or more comorbidities were present in 69.4%, 17.3%, and 13.3% of participants, respectively, and were equally distributed between study arms. Median time from hospitalization to randomization was two days (interquartile range 1 to 4.2) for placebo and two days (interquartile range 1 to 4) for proxalutamide.

FIG. 7 is a set of graphs showing the distribution of the COVID-19 8-Point Ordinal Scale in the Intention-to-Treat population at randomization, day 14, and day 28. Data are presented in the overall population (FIG. 7A) and stratified by gender (FIG. 7B).

FIG. 8 (TABLE 3). Outcomes according to treatment group in the intention-to-treat analysis. Recovery rates (reaching scores 1 or 2) over fourteen and twenty-eight days after randomization. All-cause mortality rate over twenty-eight days. (IQR=interquartile range, CI=confidence interval, HR=Hazard Ratio). Subgroup analysis per sex and per baseline score are presented with 95% confidence interval not adjusted for multiplicity.

FIG. 9 is a set of Kaplan-Meier estimates in the intention-to-treat analysis from randomization to day 28. Surviving (Panel A) and Alive Hospital Discharge (Panel B) for the overall population. These figures depict the Kaplan-Meier survival curves and alive hospital discharge within twenty-eight days for both the proxalutamide and placebo treated groups overall. The difference in the proportion surviving was evident as early as day 3 and increased over the remaining study period, which includes days after the 14-day therapy period. FIG. 9A demonstrates there was no noticeable rebound effect if therapy was completed. The difference in the proportion of alive hospital discharge was statistically significant at day 2, and increased until day 11, reaching 75%, FIG. 9B shows that patients in the proxalutamide group during the study period were 84% (95% confidence interval, 89-76%) less likely to die than patients in the control group (hazard ratio for death 0.16 (95% confidence interval, 0.11 to 0.24)). The median hospitalization time (days) was lower in the proxalutamide arm (eight, interquartile range 6-13) compared to the placebo (twelve, interquartile range 8-18).

FIG. 10 (TABLE 4). Treatment emergent adverse events (TEAE) in the patient population, per intention-to-treat analysis.

FIG. 11 is a bar graph showing the randomization/recruitment timeline.

FIG. 12 is a set of eight Kaplan-Meier estimates from randomization to Day 28. Alive Hospital Discharge and Proportion Surviving by sex and baseline ordinal scale.

FIG. 13 is a pair of graphs showing the graphical assessment of proportional-hazards assumption (hazard ratio over twenty-eight days post-randomization). Top: figure displays lines that are parallel, implying that the proportional-hazards assumption for the therapy groups was not violated. Bottom: This is confirmed by Kaplan-Meier versus predicted survival, where the observed values and predicted values are close together for the therapy groups. Proportional-hazards assumption based on Schoenfeld residuals showed a global test with P value=0.7986.

FIG. 14 (TABLE 5) lists the coronavirus disease 2019 (Covid-19) 8-point ordinal scale scores distribution and outcomes by baseline scores

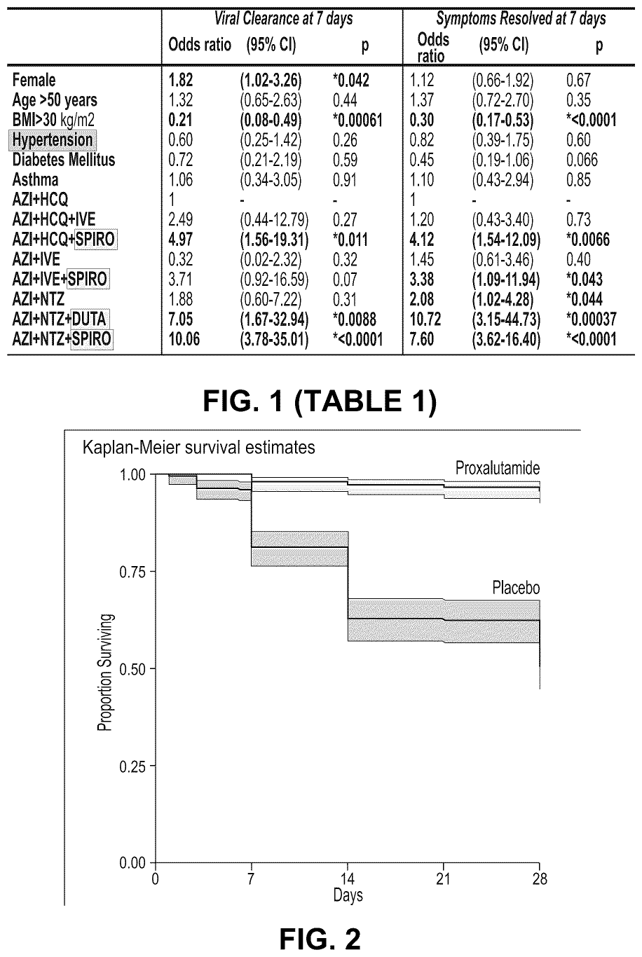

### DETAILED DESCRIPTION OF THE INVENTION

Some aspects, modes, embodiments, variations, and features of the invention are described below in various levels of detail to provide a substantial understanding of the invention.

### INDUSTRIAL APPLICABILITY

This class of medications is advantageously lifesaving to a broad group of patients presenting with sepsis from any infectious etiology, along with patients with sepsis-like conditions that include hemorrhagic shock and trauma. This invention is useful for 1.7 million Americans who develop sepsis annually.

### Definitions

For convenience, the meaning of some terms and phrases used in the specification, examples, and appended claims, are listed below. Unless stated otherwise or implicit from context, these terms and phrases have the meanings below. These definitions are to aid in describing embodiments and are not intended to limit the claimed invention. Unless otherwise defined, all technical and scientific terms have the same meaning as commonly understood by one of ordinary skill in the art to which this invention belongs. For any apparent discrepancy between the meaning of a term in the art and a definition provided in this specification, the meaning provided in this specification shall prevail.

5-alpha-reductase inhibitor (5ARi) has the biomedical art-recognized meaning. Dutasteride and finasteride are 5-alpha-reductase inhibitors. 5-alpha reductase inhibitors such as dutasteride and finasteride are prescribed for hyperandrogenic features involving dihydrotestosterone activity, such as labeled and off-label use in dermatology to treat AGA and the labeled indication use in urology for benign prostatic hyperplasia. United States Food & Drug Administration. Avodart® (dutasteride) Soft Gelatin Capsules. Highlights of Prescribing Information. Published 2008.

Acute respiratory distress syndrome (ARDS) has the medical art-defined meaning. ARDS is a type of respiratory failure characterized by rapid onset of widespread inflammation in the lungs. Symptoms include shortness of breath, rapid breathing, and bluish skin coloration. Causes may include sepsis, pancreatitis, trauma, pneumonia, and aspiration.

Comprises and comprising refer to elements, components, or steps in a non-exclusive manner, indicating that the referenced elements, components, or steps may be present, used, or combined with other elements, components, or steps. The singular terms a, an, and the include plural referents unless context indicates otherwise. Similarly, the inclusive term or should cover the term and unless the context indicates otherwise. The abbreviation e.g., means a non-limiting example and is synonymous with the term for example.

Diarylhydantoin compound has the biomedical art-recognized meaning, and includes among other chemical compounds: flutamide, bicalutamide, enzalutamide, apalutamide, and proxalutamide. These compounds competitively inhibit androgen binding, block androgen receptor nuclear translocation, and prevent their binding to DNA. Tran et al., Science, 324(5928), 787-790 (2009).

Proxalutamide is a second-generation nonsteroidal androgen receptor antagonist that is more potent than other antiandrogen compounds such as enzalutamide or bicalutamide. See Qu et al., Invest New Drugs. 2020; 38(5): 1292-1302. Clinical evidence has demonstrated that proxalutamide lowers AR expression and activity. Proxalutamide lowers the expression of ACE2.

Sepsis has the medical art-defined meaning of a life-threatening condition that arises when the body's response to infection injures its tissues and organs. Bone et al., Chest, 101, 1644-1655 (1992); Singer et al., JAMA, 315, 801-810 (February 2016). Sepsis is a life-threatening organ dysfunction due to a dysregulated host response to infection. Despite declining age-standardized incidence and mortality, sepsis remains a significant cause of health loss worldwide. Rudd et al., The Lancet, 395(10219), 200-211 (Jan. 18, 2020). Sepsis is treatable, and timely implementation of targeted interventions improves outcomes.

Treatment for sepsis has the medical-art recognized meaning. Sepsis is treatable, and timely implementation of targeted interventions improves outcomes. The Mayo Clinic informs the public that several medications are used in treating sepsis and septic shock. They include antibiotics. Broad-spectrum antibiotics, which are effective against a variety of bacteria, are usually used first. After learning the results of blood tests, a doctor may switch to a different antibiotic that's targeted to fight the specific bacteria causing the infection. They include intravenous fluids and vasopressors. Other medications include low doses of corticosteroids, insulin to help maintain stable blood sugar levels, drugs that modify the immune system responses, and painkillers or sedatives.

Treatment for COVID-19 has the medical-art recognized meaning. Corticosteroids can be therapeutic. See Prescott & Rice, Corticosteroids in COVID-19 ARDS: Evidence and hope during the pandemic. JAMA, 324, 1292-1295 (2020). Other treatments are known by persons having ordinary skill in the medical art. See Waterer & Rello, Infectious Diseases and Therapy (2020). See also Beigel et al., Remdesivir for the treatment of Covid-19—Preliminary Report. New England Journal of Medicine (2020). Several agents were explored in clinical trials as treatments for COVID-19. The most promising of these treatments are remdesivir, a viral RNA-polymerase inhibitor, remdesivir plus the Janus kinase (JAK) inhibitor, baricitinib, and dexamethasone, a glucocorticoid without mineralocorticoid effect. Beigel et al., N. Engl. J. Med., 383(19): 1813-1826 (2020); Kalil et al., N. Engl. J. Med., 384(9):795-807 (2021); Dexamethasone in Hospitalized Patients with Covid-19— Preliminary Report. N. Engl. J. Med., 384(8) (2020). Remdesivir reduce the median time to recovery of hospitalized COVID-19 patients to ten days, down from fifteen days, as observed with placebo.

Treatment for Acute respiratory distress syndrome (ARDS) has the medical-art recognized meaning. Corticosteroids can be therapeutic. See Prescott & Rice, Corticosteroids in COVID-19 ARDS: Evidence and hope during the pandemic. JAMA, 324, 1292-1295 (2020). Other treatments are known by persons having ordinary skill in the medical art.

### Materials and Methods

A person of ordinary skill in the biochemical art can use these materials and methods as guidance to predictable results when making and using the invention:

Dosage administration and compliance procedures. Proxalutamide 300 mg (3×100 mg tablets) or matching placebo was taken orally once daily with or without food, therapy was initiated soon after randomization. Treatment compliance was monitored and recorded while the patient was hospitalized. An accurate and current accounting of the dispensing of the study drug for each subject was maintained on an ongoing basis by the Investigator or delegated personnel. The number of study drug tablets dispensed to the subject was recorded on the Investigational Product Accountability Log. Patients discharged before treatment day 14 had the remaining tablets dispensed as to complete the full 14-day treatment course and were actively evaluated for compliance daily until day 14. All centers followed the same protocol.

Baseline COVID-19 8-point ordinal scale. The protocol was amended before initiation of the trial because of prevailing disease in the participating hospitals. In the original protocol, the inclusion was limited to COVID-19 8-point ordinal scale scores 3, 4, and 5, i.e., did not include patients requiring non-invasive ventilation or high flow oxygen devices. The ordinal scale is defined:
8. Death.
7. Hospitalized, on invasive mechanical ventilation or ECMO.
6. Hospitalized, on non-invasive ventilation or high flow oxygen devices.
5. Hospitalized, requiring supplemental oxygen.
4. Hospitalized, not requiring supplemental oxygen— requiring ongoing medical care (COVID-19 related or otherwise.
3. Hospitalized, not requiring supplemental oxygen— no longer requires ongoing medical care.
2. Not hospitalized, limitation on activities.
1. Not hospitalized, no limitations on activities.

Before the start of the trial, Brazil, and particularly the state of Amazonas, experienced a surge in COVID-19 resulting in an increase in severity of cases. Most hospitalized patients on the days preceding the trial were on high flow oxygen devices (COVID-19 8-point ordinal scale score 6). A decision was made to include score 6 on the day of initiating recruitment. All patients, including COVID-19 8-point ordinal scale score 6, were randomized in the same 1:1 ratio.

In three small hospital centers, the randomization strategy had to be modified due to: (1) logistic difficulties in taking the drugs to these remote hospitals and (2) limited financial resources. This was done to avoid unblinding and unmonitored sharing of blister packs among hospitals and patients. In the three sites located in the large urban city of Manaus with higher hospital certification standards, where randomization could be done as originally planned, a lower mortality rate in both arms was observed, and a lower mortality risk ratio.

The protocol was amended before initiation of the trial because of prevailing disease in the participating hospitals. In the original protocol, the inclusion was limited to COVID-19 8-point ordinal scale scores 3, 4, and 5, i.e., did not include patients requiring non-invasive ventilation or high flow oxygen devices.

Before the start of the trial, Brazil, and particularly the state of Amazonas, experienced a surge in COVID-19 resulting in an increase in severity of cases. Most hospitalized patients on the days preceding the trial were on high flow oxygen devices (COVID-19 8-point ordinal scale score 6). A decision was made to include score 6 on the day of initiating recruitment. All patients, including patients on the COVID-19 8-point ordinal scale score 6, were randomized in the same 1:1 ratio.

In three small hospital centers the randomization strategy was modified due to: (1) logistic difficulties in taking the drugs to these remote hospitals and (2) limited financial resources. This was done to avoid unblinding and unmonitored sharing of blister packs among hospitals and patients. In the three sites located in the large urban city of Manaus with higher hospital certification standards, where randomization could be done as originally planned, a lower mortality rate in both arms was observed, and a lower mortality risk ratio.

The following EXAMPLE is provided to illustrate the invention and shall not limit the scope of the invention.

### Example 1

### Proxalutamide Treatment for Hospitalized COVID-19 Patients; Double-Blinded, Placebo Control Clinical Trial of Severe Hospitalized Patients for COVID-19.

This EXAMPLE assesses the efficacy and safety of proxalutamide as a treatment for hospitalized COVID-19 male and female patients. See FIG. 2.

Disease or condition: Covid19 (severe acute respiratory syndrome).

Intervention/treatment. Proxalutamide 300 mg q.d. (once daily)

Primary outcome measures: Treatment efficacy of proxalutamide relative to standard of care arm as assessed by the COVID-19 ordinal scale [Time Frame: Day 14].

Active comparator arm: proxalutamide+standard of care.

Placebo comparator arm: Drug: standard of care.

Allocation: randomized.

Intervention model: parallel assignment.

Intervention model description: This EXAMPLE was designed as a prospective, interventional, placebo controlled, double-blinded, randomized parallel assignment investigation.

Masking description: quadruple (participant, care provider, investigator, outcomes assessor).

During the continuing SARS-COV-2 (COVID-19) pandemic, several studies reported a significant difference in the rate of severe cases between adult females and adult males (42% vs 58%). Among children under the age of fourteen, the rate of severe cases was low. Several theories were proposed to explain this difference, including cigarette smoking and lifestyle habits. No theory fits both the gender difference in severe cases and reduced risk in pre-pubescent children. Past investigation on male androgenetic alopecia (AGA) led the investigators to investigate an association between androgens and COVID-19 pathogenesis. In normal subjects, androgen expression demonstrates significant variation between men and women and between adults and pre-pubescent children.

SARS-COV-2 primarily infects type II pneumocytes in the human lung. SARS-COV-2 enters pneumocytes, by anchoring to the ACE2 cell surface receptor. Before receptor binding, viral spike proteins undergo proteolytic priming by the transmembrane protease, serine 2 (TMPRSS2). TMPRSS2 inhibition or knock down reduces ability of SARS-COV-1 (a related virus to SARS-COV-2) to infect cells in vitro. TMPRSS2 also facilitates entry of influenza A and influenza B into primary human airway cells and type II pneumocytes.

The human TMPRSS2 gene has a 15 bp androgen response element and in humans, androgens are the only known transcription promoters for the TMPRSS2 gene. In a study of androgen-stimulated prostate cancer cells (LNCaP), TMPRSS2 mRNA expression increase was mediated by the androgen receptor. Further, the ACE2 receptor, also critical for SARS-COV-2 viral infectivity, is affected by male sex hormones with higher activity found in males.

The inventors reported the results from two retrospective cohort analysis demonstrating the protective effect of 5-alpha-reductase inhibitors (5ARi) for men with COVID-19. Among seventy-seven men hospitalized with COVID-19 the investigators found among men taking 5-alpha-reductase inhibitors, 8% were admitted to an intensive care unit (ICU) compared to 58% of men not taking 5-alpha-reductase inhibitors (P=0.0015). In the cohort, 5ARis were associated with reduced risk for intensive care unit admissions. RR 0.14 (95% CI: 0.02-0.94). The frequency of COVID-19 symptoms was drastically reduced for men using 5-alpha-reductase inhibitors in an outpatient setting. A statistically significant (p<0.05) reduction in the frequency of 20 of the twenty-nine clinical symptoms was observed in androgenetic alopecia men using 5ARis compared to androgenetic alopecia men not using 5-alpha-reductase inhibitors. For example, 38% and 2% of men presented with low-grade fever, 60% and 6% with dry cough, and 88% and 15% reported anosmia in the non-5ARi and 5ARi groups, respectively.

One limitation of 5ARis is the time course required to achieve systemic DHT reductions. The investigators explored the use of a second generation androgen receptor antagonist proxalutamide as a means for rapid reduction in AR activity. Proxalutamide (GT0918) demonstrates a dual mechanism of action. It is highly effective in inhibiting AR and exhibiting pharmacological effects of inducing the down-regulation of AR expression; the mechanism that is not present in bicalutamide and enzalutamide. Because of the dual mechanism of action, this should be a more effective and less toxic second-generation anti-androgen drug therapy. Clinical evidence demonstrated that proxalutamide lowers AR expression and activity. Proxalutamide lowers the expression of ACE2. Both medications would be beneficial for preventing SARS-COV-2 entry into lung cells. None of the 5ARis currently approved by the US FDA were tested in phase I studies in women, and so are not recommended for women. Phase I studies for proxalutamide were successfully completed in both men and women.

### Example 2

### Proxelutamide Treatment for Hospitalized COVID-19 Patients-Preliminary Report

Preliminary Outcome Measure: Treatment efficacy of proxalutamide relative to placebo arm as assessed by the COVID-19 ordinal scale [Time Frame: Day 14].

TABLE 6

| | Active | | Placebo | |
|---|---|---|---|---|
| | (n = 294) | | (n = 296) | |
| Male:Female (% male) | 167:127 | (56.8%) | 171:125 | (57.8%) |
| Age Mean ± SD Median (IQR) | 53.5 ± 14.5 | | 53.0 ± 15.5 | |
| | 52 (21) | | 53 (25) | |
| Clinical status on Day 0 | Oxygen use (%) | | Oxygen use (%) | |
| Non-invasive ventilation (NIV) or | 97.3% | (286/294) | 95.9% | (284/296) |
| high flow oxygen (HFO) (%) | 69.0% | (203/294) | 95.9% (284/296) | |
| | | | 66.6% (197/296) | |
| Mortality (%) Day 14 | 3.7% | (11/294) | 47.6% | (141/296) |
| Hospital Length Stay (Days) | | | | |
| Mean ± SD | 6.3 ± 5.3 | | 15.4 ± 9.3 | |
| Median (IQR) | 5.0 | (4.0) | 14.0 | (10.0) |
| New mechanical ventilation (MV) | | | | |
| or Death (%) | | | | |
| Day 14 | 4.4% | (13/294) | 52.7% | (156/296) |
| HFO, NIV, or MV use, or death (%) | | | | |
| Day 14 | 4.8% | (14/294) | 57.8% | (171/296) |
| No HFO or NIV use (%) | | | | |
| Day 14 | 95.2% | (280/294) | 42.2% | (125/296) |
| No oxygen use (%) | | | | |
| Day 14 | 92.5% | (272/294) | 33.8% | (100/296) |
| Discharged (%) | | | | |
| Day 14 | 89.1% | (262/294) | 32.8% | (97/296) |
| Full clinical remission (%) | | | | |
| Day 14 | 75.9% | (223/294) | 8.4% | (25/296) |
| Improvement in clinical status - | | | | |
| Ordinary scale (days) | | | | |
| One point | | | | |
| Mean ± SD | 3.0 ± 5.5 | | 18.7 ± 11.3 | |
| Median (IQR) | 1.0 | (2.0) | 28.0 | (21.0) |
| Two points | | | | |
| Mean ± SD | 5.5 ± 7.1 | | 20.8 ± 9.9 | |
| Median (IQR) | 3.0 | (6.0) | 28.0 | (14.0) |

### Example 3

Accelerated Viral Clearance and Symptom Resolution in Symptomatic COVID-19 Outpatients Treated with Anti-Androgens

Summary A short course of antiandrogens was associated with improved virologic and clinical outcomes in COVID-19 outpatients. By modifying host factors, antiandrogens increased the viral clearance rate by 320%, and symptom resolution rate by 70% in the first week of therapy.

SARS-COV-2 cell entry involves an androgen-regulated protease, TMPRSS2. Antiandrogens could provide a new approach to COVID-19 management. The inventors evaluated the effects of antiandrogens initiated upon diagnosis on the nasopharyngeal viral clearance and symptom resolution.

Methods. Prospective cohort of 585 [475 final] outpatients treated with off-label regimens in Brazil from June-August 2020. Control group was usual care therapeutic combinations based on prevailing practice at the time: azithromycin 500 mg/day/five days with either hydroxychloroquine 400 mg/day/five days, ivermectin 0.2 mg/kg/day/three days, or nitazoxanide 500 mg/twice daily/six days. Antiandrogen therapy was spironolactone 100 mg twice daily (men and women) or dutasteride 0.5 mg/day (men) until symptom resolution. Primary outcome was nasopharyngeal RT-PCR-SARS-COV-2 clearance ratio at seven days. Secondary outcome was symptom resolution ratio at seven days.

Findings. Viral clearance at seven days was 37% (antiandrogen) versus 9% (control), P<0.001. The viral clearance ratio was 4.2 (95% confidence interval 2.7-6.7), a 320% increase in viral clearance rate, 95% confidence interval (170-570%). The viral clearance ration was 4.2 (95% confidence interval [CI] 2.7-6.7), favoring antiandrogen therapy. Symptom resolution was 79% (antiandrogen) versus 48% (control), P<0.001. The symptom resolution ratio was 1.7; 95% confidence interval (1.4-1.9), a 70% increase in symptom resolution rate, 95% confidence interval (40-90%).

Introduction. Androgens augment SARS-COV-2 infectivity by promoting the expression of transmembrane protease (TMPRSS2) that primes the spike viral entry protein. See Clinckemalie et al., Molecular Endocrinology, Volume 27, Issue 12, pages 2028-2040 (Dec. 1, 2013) to support the idea that TMPRSS2 is positively regulated by androgens. See also Letko et al., Nature Microbiology, Volume 5, pages 562-569 (Feb. 24, 2020) to support the idea that TMPRSS2 promotes virus.

Several clinical observations support a role of androgens in exacerbating COVID-19. Women with polycystic ovary syndrome have an increased risk of COVID-192 and have more symptoms than non-hyperandrogenic counterparts. Subramanian et al., Eur. J. Endocrinol., 184(5), 637-645 (2021); Cadegiani et al., J. Eur. Acad. Dermatology Venereol., 35(2), (2021).

Androgenetic alopecia (AGA), a clinical sign of increased androgen hormone expression was present in most COVID-19 hospitalized patients and the severity of androgenetic alopecia is associated with worse COVID-19 outcomes. Wambier et al., J. Am. Acad. Dermatol., 83(2), 680-682 (2020); Salazar Arenas et al., Le Infez Med. 29(1), 37-45 (2021); Wambier et al., J. Am. Acad. Dermatol. (July 2020); & Lee, Yousaf, Fang, & Kolodney. J. Am. Acad. Dermatol. (July 2020). These findings support more detailed clinical investigations into antiandrogen therapy.

Several existing, approved medications could be repurposed for COVID-19. The mineralocorticoid receptor antagonist spironolactone is a known androgen receptor antagonist. Dhurat et al., Dermatol. Ther., 34(1) (2021). 5-alpha reductase inhibitors such as dutasteride and finasteride are prescribed for hyperandrogenic features involving dihydrotestosterone activity, such as labeled and off-label use in dermatology to treat androgenetic alopecia and the labeled indication use in urology for benign prostatic hyperplasia. Both spironolactone and dutasteride were found to decrease the expression of TMPRSS2 and angiotensin converting enzyme 2 (ACE2) in men, making them candidates for generic, low-cost COVID-19 therapy. Samuel et al., Cell Stem Cell, 27(6), 876-889.e12 (2020).

The inventors previously showed that long-term users of anti-androgens have less severe COVID-19. When hospitalized due to COVID-19, those on antiandrogens for over 6 months had reduced chances of being admitted to an intensive care unit. Goren et al., J. Eur. Acad. Dermatology Venereol. (2020). Among men with diagnosis of AGA, dutasteride users had less symptomatic COVID-19 disease in the outpatient setting. McCoy et al., J. Eur. Acad. Dermatol Venereol., 35(4), e243-e246 (2021). The objective of this EXAMPLE was to investigate whether initiating antiandrogens at the time of diagnosis altered the course of COVID-19 measured by SARS-COV-2 viral clearance and symptom resolution during the first seven days of therapy.

Methods. This prospective cohort EXAMPLE used a dataset stripped of protected health information that is publicly available. Cadegiani, Pre-RCT AndroCoV Trial—COVID-19 public dataset 2020-10-31. OSF. Published 2020. The data are from a consecutive series of >18-year-old patients followed from Jun. 15, 2020, to Aug. 30, 2020, at a reference outpatient COVID-19 center in Brasilia, Brazil. Patients with a COVID-19 diagnosis within a week of symptom onset and confirmed by nasopharyngeal reverse transcriptase polymerase chain reaction test for SARS-COV-2 were included in the analysis. The inventors excluded from the analysis asymptomatic patients, and those with previously using spironolactone or 5-alpha reductase inhibitors, and therapy modalities with scarce representation (five or fewer patients, i.e., patients that took azithromycin with hydroxychloroquine, ivermectin and antiandrogens, or azithromycin with hydroxychloroquine and nitazoxanide, or azithromycin with hydroxychloroquine with nitazoxanide and antiandrogens. Patients were also excluded if baseline clinical or radiological findings showed severe acute respiratory distress, including oxygen saturation (SatO₂) below 92%, or over 25% of lung involvement in a chest computed tomography (CT) scan. Patients with glucocorticoid use in the previous seven days were excluded. No patient was hospitalized, died, or was lost to follow-up.

The usual care was offered with off-label medications being used at that time: azithromycin (AZI) 500 mg/day for five days plus either hydroxychloroquine (HCQ) 400 mg/day for five days, ivermectin (IVE) 0.2 mg/kg/day for three days, or nitazoxanide (NTZ) 500 mg/twice daily for six days.

Antiandrogen therapy was also adopted as a supplement to the usual care therapeutic regimen based on earlier retrospective findings and was already being offered to patients with clinical signs of hyperandrogenism, such as baldness or hirsutism, or high body mass index (BMI). See Cadegiani et al., J. Eur. Acad. Dermatology Venereol., jdv. 17004 (November 2020); McCoy et al., J. Eur. Acad. Dermatology Venereol., jdv. 17021 (November 2020). See also Clinckemalie et al., Molecular Endocrinology, Volume 27, Issue 12, pages 2028-2040 (Dec. 1, 2013) to support the idea that TMPRSS2 is positively regulated by androgens and that TMPRSS2 promotes virus. See further Letko et al., Nature Microbiology, Volume 5, pages 562-569 (Feb. 24, 2020) to show that androgens promote infection. This observational study was distinct from a double-blinded placebo-controlled trial for antiandrogens in men (NCT04729491). When recruitment was initiated for this clinical trial, some individuals declined enrollment in a blinded study and preferred taking antiandrogen therapy on an open-label basis.

The outcomes of men and women prescribed antiandrogen therapy outside of the clinical trial. The antiandrogen group consisted of patients who were offered either spironolactone 100 mg twice a day (females and males) or dutasteride 0.5 mg per day (males only) for up to thirty days or until full recovery from symptoms. While patients that declined or were not offered antiandrogens were considered the control group.

All patients underwent questionnaire screening and were followed for COVID-19 symptoms based on the presence of at least one symptom from the upper-respiratory tract, musculoskeletal, cardiovascular, gastrointestinal, or neurological systems, or any unspecific symptom, such as fever, shortness of breath, dry cough, anosmia or ageusia. All patients agreed and signed an informed consent authorizing use of deidentified clinical data for future studies and consented to the off-label prescription of the medications. All patients underwent baseline nasopharyngeal PCR testing that was repeated every seven days after initiating therapy until a negative test result. All patients were followed for at least sixty days. Other relevant clinical data such as body mass index, age, concomitant medications, and previous medical history were also recorded on the dataset of 585 cases.

The primary outcome was viral clearance ratio at seven days of therapy initiation, defined by the relative rates of negative RT-PCR-SARS-COV-2 swab results at the seven days cut-off. The timepoint of seven days was chosen because all patients underwent this post-therapy RT-PCR-SARS-COV-2 test within the first week of therapy. Also, and viral clearance at day 7 was used for comparison among studies in a living systematic review on drug treatments for COVID-19. See Update to living systematic review on drug treatments for Covid-19. BMJ., n858 (March 2021). Patient-reported resolution of symptoms within seven days of therapy initiation was a secondary outcome, though anosmia and ageusia were excluded because of the recognized persistence of these symptoms after COVID-19 resolution. Meng, Deng, Dai, & Meng, Am. J. Otolaryngol., 41(5), 102581 (2020). Analyzed risk factors included sex, age, body mass index, diagnosis of diabetes, hypertension, and asthma.

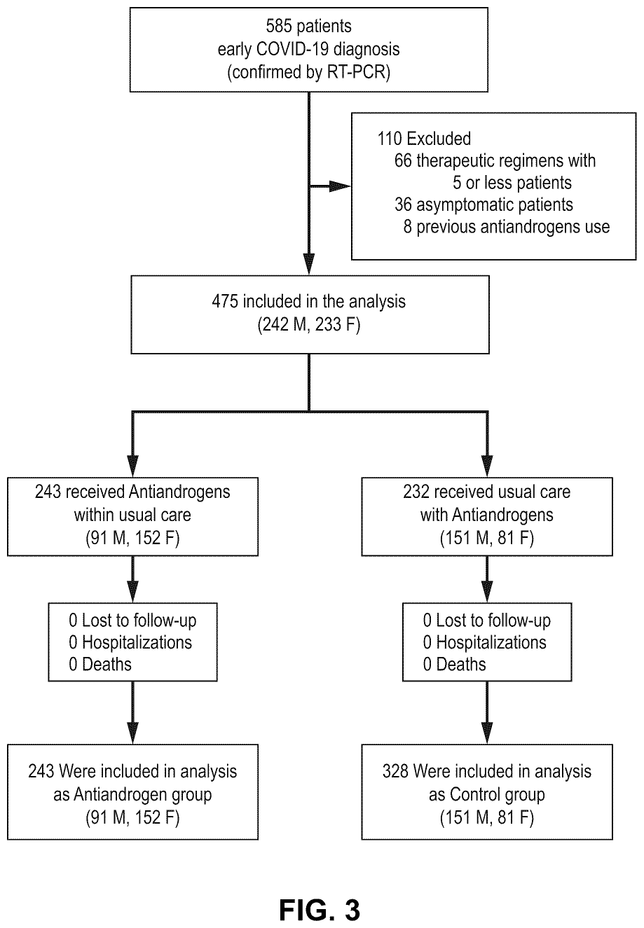

After exclusions, 475 participants (233 females and 242 males) were analyzed. A total of 243 patients (51.2%) were in the antiandrogen group, with either spironolactone (n=215) or dutasteride (n=28). The remaining 232 patients received usual care without antiandrogens and were considered the control group. See FIG. 3.

The statistical analysis measured risk ratios for viral clearance and symptom resolution. Fisher's exact test was performed for categorical variables and with P<0.05 considered significant. Multivariate logistic regression models were performed to control for baseline risk factors. A post-hoc power estimation based on a control group of 232 patients with viral recovery rate of 9% in seven days, a total sample size of 475 patients would confer an estimated power of 81.96% to detect a recovery ratio of 2 (antiandrogen group with 18% recovery rate), and an estimated power of 99% to detect a recovery ratio >3). The statistical analyses were performed using R (version 4.0.1) and Stata/SE 17.0 for Mac (StataCorp, TX USA).

Results. Patient characteristics, including the baseline symptoms, risk factors, and usual care medications are shown in TABLE 7, below. The overall median duration of symptoms was three days interquartile range (IQR) of three to four days when the treatment was initiated, with similar duration between the antiandrogen group (three days, interquartile range: two to four days) and the control group (four days, interquartile range: three to five days). Symptoms at baseline were relatively similar between groups, except for these differences: 43% of the control group versus 34% of the antiandrogen group had respiratory symptoms, 19% of the control group versus 24% of the antiandrogen group had anosmia or ageusia, and muscular weakness was present at baseline in 31% of the patients prescribed antiandrogens versus 26% in the control group. See TABLE 7, below.

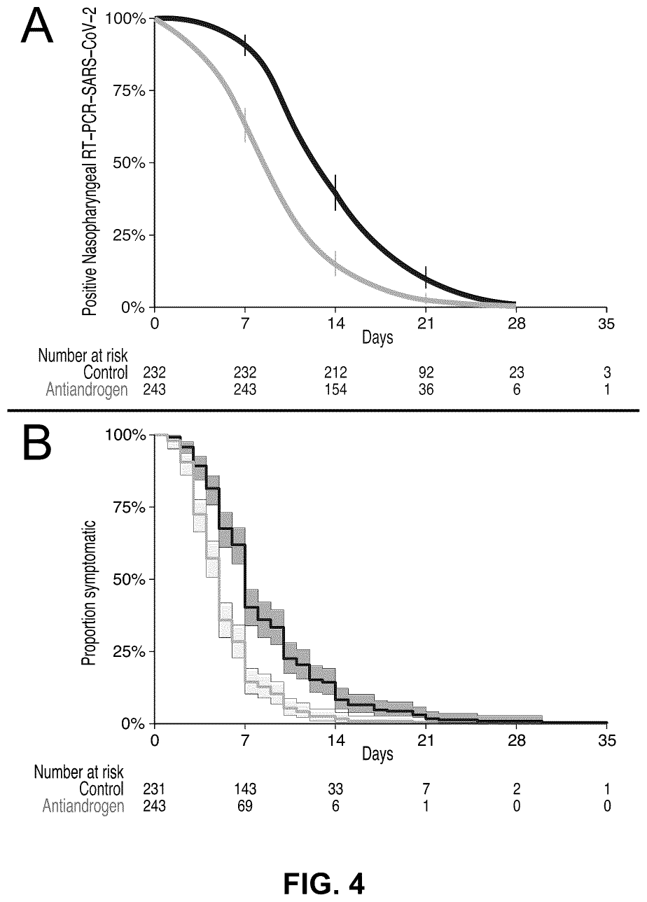

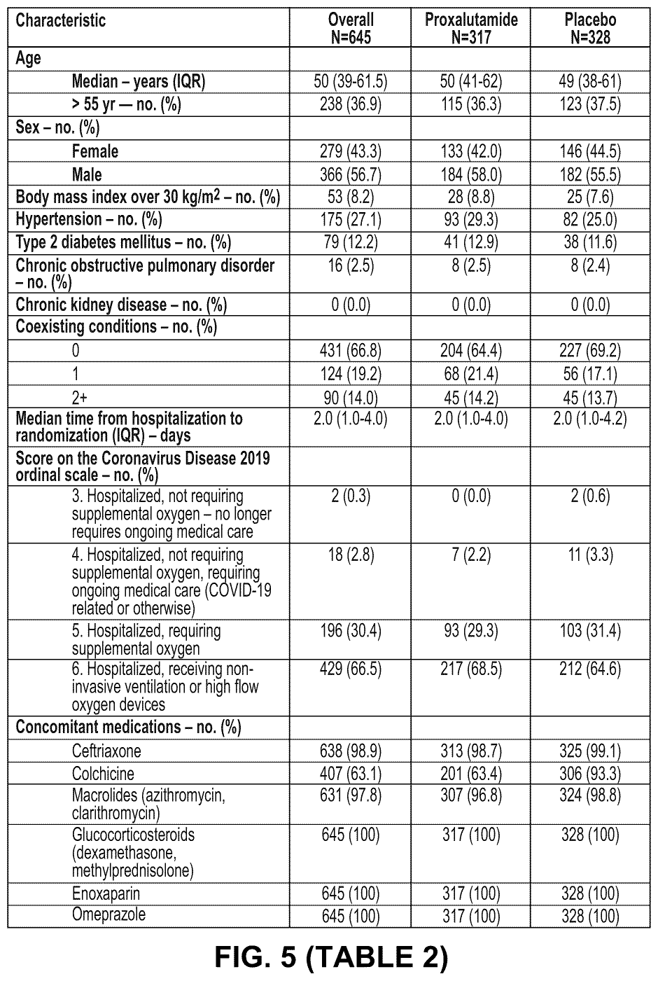

Antiandrogen therapy with either spironolactone or dutasteride was administered for up to thirty days, or until symptom resolution. The median duration of symptoms after treatment initiation in the antiandrogen group was five days, interquartile range: three to seven days, while the median duration of symptoms in the control group was seven days, interquartile range: five to ten days. The symptoms resolved before nasopharyngeal viral clearance, as shown in Kaplan-Meier curves that depict the effects of antiandrogens in viral clearance and symptoms. See FIG. 4.

Overall, antiandrogen therapy group had a viral clearance rate of 37% at seven days, compared to 9% of the control group (FIG. 4A), P<0.001; the viral clearance ratio was 4.2 (95% confidence interval [CI] 2.7-6.7), TABLE 8. This effect occurred in males: 34% (antiandrogens) versus 7% (control), P<0.001; and females, 38% (antiandrogens) versus 11% (control), P<0.001. Females were more likely to have viral clearance within seven days, 29% versus 17% (males), P=0.003. Spironolactone was prescribed mostly for females. Only male patients used dutasteride.

When evaluating specific antiandrogens, initiating spironolactone at the time of COVID-19 diagnosis was associated with more rapid viral clearance: 34% (spironolactone) versus 7% (control). The benefits of spironolactone were seen regardless of the other medications added: ivermectin: 26% (spironolactone) versus 2% (control); or nitazoxanide: 42% (spironolactone) versus 11% (control). Dutasteride was associated with increased viral clearance among nitazoxanide male users: 21% versus 8.6% (control). A trend to faster clearance was observed in men receiving spironolactone (40%) compared to dutasteride (21%). Under multivariate analysis, spironolactone and dutasteride use were associated with enhanced viral clearance within seven days particularly when used with nitazoxanide, odds ratio (OR)=10.1; 95% confidence interval (3.78-35.01) and OR 7.1; 95% confidence interval (1.7-32.9), respectively, TABLE 9.

Antiandrogen therapy initiation was also associated with improved symptom resolution. By day 7 of therapy, 79% of those who received antiandrogens had recovered from their symptoms versus 48% in controls (FIG. 4B), P<0.001, overall symptom resolution ratio was 1.7; 95% confidence interval (1.4-1.9), so the antiandrogen group had 40 to 90% higher chances of resolving symptoms (excluding smell and taste) compared to the control group.

Upon multivariate analyses that controlled for patient baseline characteristics, across all patients, females had higher chances of viral clearance at seven days, odds ratio (OR)=1.82, P=0.042 while patients with body mass index (BMI)>30 kg/m2 had lower chances of viral clearance, OR=0.21, P<0.001. Antiandrogens use was associated with faster improvement in symptoms (OR>3) in all regimens, TABLE 9.

Discussion The results of this observational, open label, EXAMPLE showed that initiating antiandrogen therapy upon COVID-19 diagnosis led to virologic and clinical improvements. Early antiandrogen therapy increased the rates of nasopharyngeal viral clearance by RT-PCR-SARS-COV-2 within the first 7 days by 320%. Antiandrogens were associated with a relative improvement in COVID-19 symptoms during the first week of therapy of 70%. The impact of the antiandrogens was seen independent of the background usual care regimen administered or other patient characteristics, including gender. There were no adverse events noted with the administration of spironolactone or dutasteride for this short duration.

Viral clearance determined by repeat PCR testing was an outcome measure in multiple studies. More rapid viral clearance was previously detected with nitazoxanide 500 mg three times a day for five days. Rocco et al., Early use of nitazoxanide in mild Covid-19 disease: randomised, placebo-controlled trial. Eur Respir J. (January 2021). In this EXAMPLE, nitazoxanide was not associated faster clearance in a lower dose regimen (two times a day for 6 days) used combined with azithromycin. The combination of this regimen with antiandrogens showed an association with faster clearance rates in a multivariate analysis. A multidrug combination of nitazoxanide and hydroxychloroquine, but not azithromycin with hydroxychloroquine, shortened the period to SARS-COV-2 viral clearance compared to placebo in the outpatient adult setting. Elalfy et al., J. Med. Virol. 93(5), 3176-3183 (2021); Johnston et al., EClinicalMedicine, 33, 100773 (2021).

A lower dose of nitazoxanide at 300 mg extended release tablets for four days did not reduce the proportions of positive SARS-COV-2 or viral load at days 4 or 10 in a randomized outpatient study of early treatment. Rossignol et al., medRxiv., 2021.04. 19.21255441 (January 2021).

Antiandrogen therapy was associated with viral clearance in a greater proportion of patients at day 7.

Patient-reported-outcomes and symptoms have recently been used in clinical trials. Neither nitazoxanide, hydroxychloroquine (with or without azithromycin), nor ivermectin had patient-reported effects on symptom resolution compared to placebo. In this EXAMPLE, initiation of antiandrogen therapy was associated with increased symptom resolution at seven days.

The results described the EXAMPLE supports antiandrogen therapy in COVID-19. In an inpatient randomized control trial (RCT) in male patients aged fifty years or older, 5 mg daily of finasteride reduced inpatient mortality, though without achieving statistical significance. Zarehoseinzade et al., MJIRI. 35(1), 232-237 (2021). Zarehoseinzade et al., MJIRI. 35(1), 232-237 (2021). Finasteride has a short half-life of six hours, compared to five weeks for dutasteride. Wambier, Pereira, Prado Júnior, & Foss, An Bras Dermatol. 87(4), 590-595 (2012). Dutasteride could have a greater impact on COVID-19, since the results from a placebo-controlled randomized control trial of dutasteride 0.5 mg per day in male patients showed increased viral clearance at seven days from 11.8% (placebo group) to 64.3% (dutasteride). Cadegiani, McCoy, Gustavo Wambier, & Goren. Cureus (February 2021). More robust findings were detected in a placebo-controlled randomized control trial of another antiandrogen, proxalutamide, a potent androgen receptor blocker, demonstrated increased viral clearance at seven days in both men (from 21% [placebo] to 81% [proxalutamide]) and women (from 38% [placebo] to 85% [proxalutamide]). Cadegiani et al., Cureus (February 2021). The results of a randomized, placebo-controlled trial of proxalutamide 200 mg for up to seven days in a trial that enrolled 268 men showed that 30-day hospitalization rate was 2.2% in men taking proxalutamide compared to 26% in those taking placebo, P<0.001, a reduction of 91% of the risk of hospitalization compared to usual care. McCoy et al. Front Med. (2021). See also the case of a young bodybuilder developing severe COVID-19 symptoms while using the dihydrotestosterone analogue oxandrolone as an anabolic steroid, who had striking symptom resolution and laboratory improvement within twenty-four hours of starting proxalutamide Cadegiani, Lin, Goren, & Wambier, BMJ Case Rep., 14(2), e241572 (2021).

Canrenone, an active metabolite of spironolactone, was associated with reduced fatalities in an intensive care observational series of cases with hypertension or hypokalemia. Vicenzi et al., J Clin Med., 9(9) (2020). The accumulating evidence taken together suggests a range of antiandrogens are beneficial in COVID-19.

The EXAMPLE design was opportunistic, taking advantage of patients who elected to try off-label use of antiandrogens outside of a clinical trial. No other substantial differences were seen between antiandrogen and control groups besides gender (TABLE 7), which was among the risk factors controlled under the multivariate analyses (TABLE 9), Muscle symptoms and anosmia had higher incidence in the antiandrogen group. Anosmia was not accounted in the symptom resolution.

Gender distribution was different between the two antiandrogen groups since women were not prescribed dutasteride (TABLE 7). This patient characteristic was controlled for in the multivariate analysis (TABLE 9). Respiratory symptoms were more prevalent in the control group. Muscle symptoms, anosmia, and symptoms like fever and headache were more prevalent in the antiandrogen group.

An unknown variable may be asymmetrically distributed between the groups, such as willingness to be treated with antiandrogens. Though anti-androgen effects were seen across individuals receiving different combinations of medicines, there was also concomitant use of other medications with potential antiviral activity. The EXAMPLE's observational design did not account for potential bias in selecting these alternative therapies. Azithromycin plus hydroxychloroquine may have been avoided in patients already receiving other medications that prolong QTc.

Conclusion. Spironolactone and dutasteride, antiandrogens that are widely used and available worldwide as generic medications, increased the viral clearance rates by 320% and resolution of COVID-19 symptoms by 70% in the outpatient setting in the first week of therapy. Investigation of antiandrogen monotherapy is warranted.

TABLE 7
Baseline Characteristics

| | Overall | | Control | | Antiandrogen | |
|---|---|---|---|---|---|---|
| Characteristic | N = 475 | | N = 232 | | N = 243 | |
| Sex - no. (%) | | | | | | |
| Females | 233 | (49) | 81 | (35) | 152 | (63) |
| Male | 242 | (51) | 151 | (65) | 91 | (37) |
| Duration of symptoms - days (IQR) | 3 | (3-4) | 4 | (3-5) | 3 | (2-4) |
| Symptoms at baseline visit - no. (%) | | | | | | |
| Anosmia/Ageusia | 103 | (22) | 45 | (19) | 58 | (24) |
| Muscular/weakness symptoms (i.e.: | 135 | (28) | 60 | (26) | 75 | (31) |
| dengue-like presentation) | | | 99 | (43) | 82 | (34) |
| Respiratory symptoms (i.e.: cough, | 181 | (38) | | | | |
| sore throat) | | | | | | |
| Gastrointestinal symptoms (i.e.: | 60 | (13) | 29 | (13) | 31 | (13) |
| diarrhea, vomiting) | | | | | | |
| Mixed (>1 of the above) | 58 | (12) | 29 | (13) | 29 | (12) |
| Non-specific (i.e., fever, headache) | 97 | (20) | 41 | (18) | 56 | (23) |
| Asymptomatic | 0 | (0) | 0 | (0) | 0 | (0) |
| Risk Factors - no. (%) | | | | | | |
| Age >50 years | 118 | (25) | 61 | (26) | 57 | (23) |
| Body Mass Index >30 kg/m² | 86 | (18) | 48 | (21) | 38 | (16) |
| Hypertension | 80 | (17) | 38 | (16) | 42 | (17) |
| Diabetes Mellitus | 45 | (10) | 23 | (10) | 22 | (9) |
| Asthma | 32 | (7) | 16 | (7) | 16 | (7) |
| COVID-19 usual care | | | | | | |
| medications - no. (%) | | | | | | |
| Azithromycin | 475 | (100) | 232 | (100) | 243 | (100) |
| Hydroxychloroquine | 118 | (25) | 77 | (33) | 41 | (17) |
| Ivermectin | 87 | (18) | 64 | (28) | 23 | (9) |
| Nitazoxanide | 293 | (62) | 114 | (49) | 28 | (12) |
| Spironolactone | 215 | (45) | 0 | (0) | 215 | (88) |
| Dutasteride | 28 | (6) | 0 | (0) | 28 | (12) |

TABLE 8
Outcomes: viral clearance rates (nasopharyngeal RT-PCR-SARS-CoV-2) at seven days
and recovery from symptoms rates (except anosmia and ageusia) within seven days.

| | | | | | Anti- | | | Number |
|---|---|---|---|---|---|---|---|---|
| | Overall | | Control | | androgen | | Risk Ratio | needed to |
| Characteristic | N = 475 | | N = 232 | | N = 243 | | (95% CI) | treat (95% CI) |
| Viral clearance - | 109 | (23) | 20 | (9) | 89 | (37) | 4.2 (2.7-6.7) | 4 (5-3) |
| no. (%) | | | | | | | | |
| Recovery from | 303 | (64) | 111 | (48) | 192 | (79) | 1.7 (1.4-1.9) | 3 (4-3) |
| symptoms - no. (%) | | | | | | | | |
| Days for recovery | 7 | (7-14) | 7 | (5-10) | 5 | (3-7) | — | — |
| from symptoms- | | | | | | | | |
| median (IQR) | | | | | | | | |

TABLE 9
Multivariate logistic regression of patient characteristics and treatment modalities,
for nasopharyngeal RT-PCR-SARS-COV-2 viral clearance and resolution of symptoms
(except anosmia, ageusia) within 7 days of therapy initiation, N = 475.

| | | | Viral Clearance | | | Resolution of Symptoms | | |
|---|---|---|---|---|---|---|---|---|
| Characteristic | | | Odds ratio | | | Odds ratio | | |
| Baseline risk factor | No. (%) | | (95% CI) | | P | (95% CI) | | P |
| Female | 233 | (49) | 1.82 | (1.02-3.26) | *0.042 | 1.12 | (0.66-1.92) | 0.67 |
| Age >50 years | 118 | (25) | 1.32 | (0.65-2.63) | 0.44 | 1.37 | (0.72-2.70) | 0.35 |
| Body Mass Index | 86 | (18) | 0.21 | (0.08-0.49) | *<0.001 | 0.30 | (0.17-0.53) | *<0.001 |
| >30 kg/m² | | | | | | | | |
| Hypertension | 80 | (17) | 0.60 | (0.25-1.42) | 0.26 | 0.82 | (0.39-1.75) | 0.60 |
| Diabetes Mellitus | 45 | (10) | 0.72 | (0.21-2.19) | 0.59 | 0.45 | (0.19-1.06) | 0.07 |
| Asthma | 32 | (7) | 1.06 | (0.34-3.05) | 0.91 | 1.10 | (0.43-2.94) | 0.85 |
| Azithromycin with: | 475 | (100) | | | | | | |
| Hydroxychloroquine | 54 | (11) | 1 | | — | 1 | | — |
| Ivermectin | 41 | (9) | 0.32 | (0.02-2.32) | 0.32 | 1.45 | (0.61-3.46) | 0.40 |
| Nitazoxanide | 114 | (24) | 1.88 | (0.60-7.22) | 0.31 | 2.08 | (1.02-4.28) | *0.04 |
| Hydroxychloroquine | 23 | (5) | 2.49 | (0.44-12.79) | 0.27 | 1.20 | (0.43-3.40) | 0.73 |
| and Ivermectin | | | | | | | | |
| Ivermectin and | 23 | (5) | 3.71 | (0.92-16.59) | 0.07 | 3.38 | (1.09-11.94) | *0.04 |
| Spironolactone | | | | | | | | |
| Hydroxychloroquine | 41 | (9) | 4.97 | (1.56-19.31) | *0.011 | 4.12 | (1.54-12.09) | *0.007 |
| and Spironolactone | | | | | | | | |
| Nitazoxanide and | 28 | (6) | 7.05 | (1.67-32.94) | *0.009 | 10.72 | (3.15-44.73) | *<0.001 |
| Dutasteride | | | | | | | | |
| Nitazoxanide and | 151 | (32) | 10.06 | (3.78-35.01) | *<0.001 | 7.60 | (3.62-16.40) | *<0.001 |
| Spironolactone | | | | | | | | |

### Example 4

### Efficacy of Proxalutamide in Hospitalized COVID-19 Patients: A Randomized, Double-Blind, Placebo-Controlled, Parallel-Design Clinical Trial

Conclusions. Hospitalized COVID-19 patients receiving treatment with proxalutamide had a 128% higher recovery rate than those treated with placebo at day 14. All-cause mortality was reduced by 77.7% over twenty-eight days.

Background Severe acute respiratory syndrome coronavirus 2 (SARS-CoV-2) infectivity is mediated by the androgen-promoted protease, transmembrane protease, serine 2 (TMPRSS2). Treatment with proxalutamide, a non-steroidal androgen receptor antagonist, accelerates viral clearance and clinical remission in outpatients with coronavirus disease 2019 (COVID-19) compared to placebo. The effects in hospitalized COVID-19 patients were unknown.

Methods. Men and women hospitalized but not requiring mechanical ventilation were randomized (1:1 ratio) to receive 300 mg of proxalutamide per day or placebo for 14 days. The study was conducted at eight sites in the state of Amazonas, Brazil. The primary outcome measure was the clinical status (8-point ordinal scale) at 14-days post-randomization. The primary efficacy endpoint was the 14-day recovery ratio (alive hospital discharge [scores 1, 2]).

Results. A total of 645 patients were randomized (317 received proxalutamide, 328 placebo) and underwent intention-to-treat analysis. The 14-day median ordinal scale score in the proxalutamide group was 1 (interquartile range [IQR]=1-2) versus 7 (interquartile range=2-8) for placebo, P<0.001. The 14-day recovery rate was 81.4% for proxalutamide and 35.7% for placebo (recovery ratio, 2.28; 95% confidence interval 1.95-2.66 [P<0.001]). The 28-day all-cause mortality rate was 11.0% for proxalutamide versus 49.4% for placebo (hazard ratio, 0.16; 95% confidence interval 0.11-0.24). The median post-randomization time to recovery was 5 days (interquartile range=3-8) for proxalutamide versus 10 days (interquartile range=6-15) for placebo. Hospitalized COVID-19 patients not requiring mechanical ventilation receiving proxalutamide had a 128% higher recovery rate than those treated with placebo. All-cause mortality was reduced by 77.7% over 28 days. (ClinicalTrials.gov number, NCT04728802).

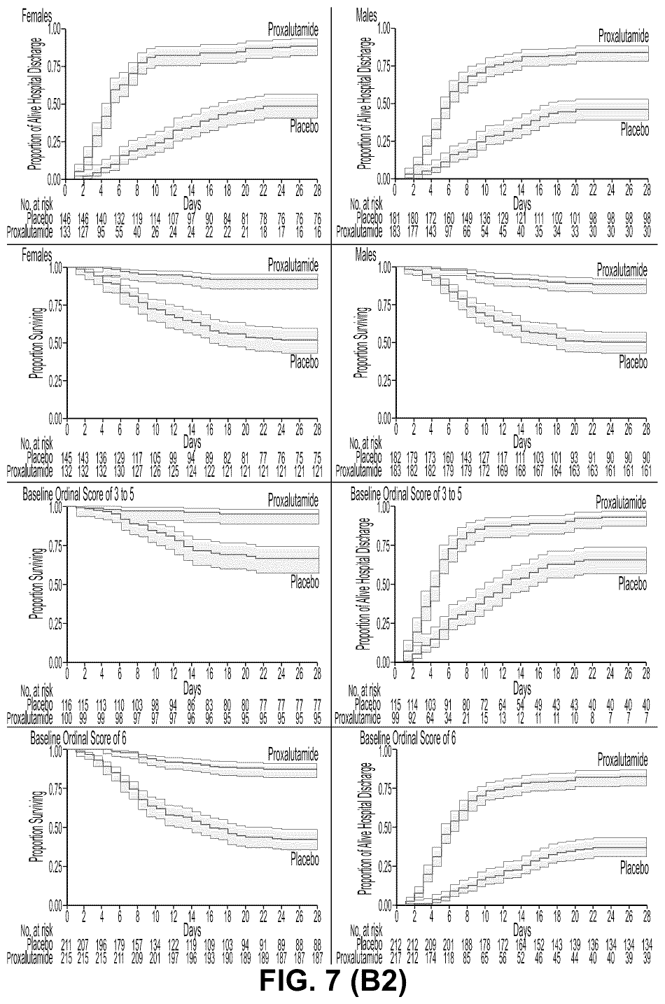

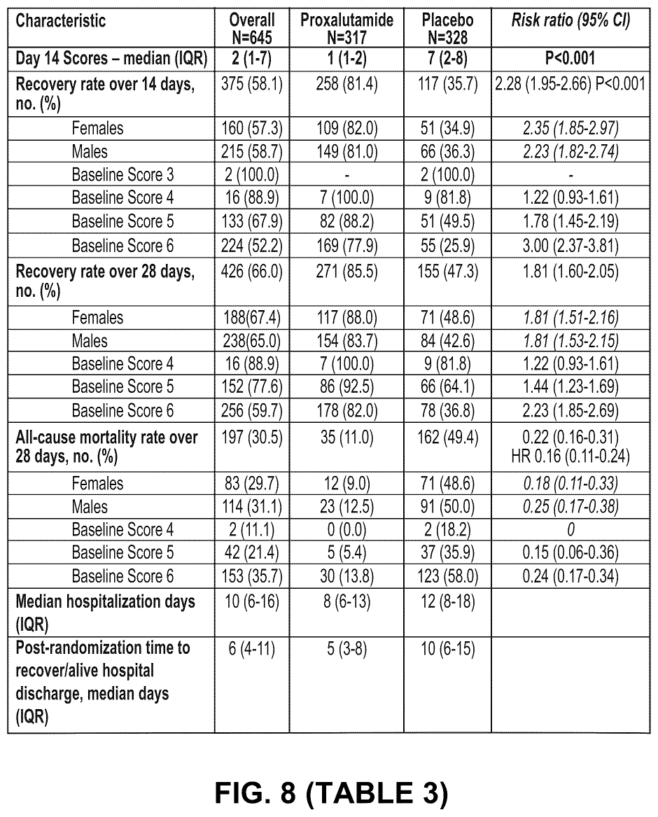

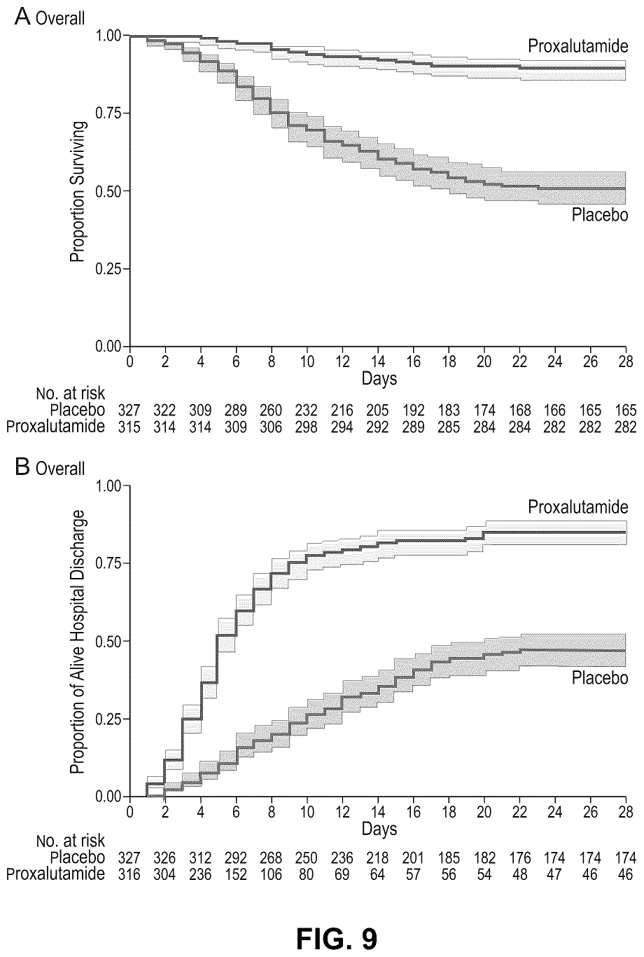

Primary outcome. At the 14-day timepoint, a lower score distribution was observed for the proxalutamide group than in the placebo group. See FIG. 7A. The median score in the proxalutamide group was 1 (interquartile range 1 to 2) versus 7 (interquartile range 2 to 8) for placebo (P<0.001). See FIG. 8 (TABLE 3). The overall 14-day recovery rate for placebo of 35.7% (95% confidence interval 30.7 to 40.1) was lower than for proxalutamide (81.4%; 95% confidence interval 76.7 to 85.3), P<0.001. The 14-day recovery ratio was 2.28 (95% confidence interval 1.95 to 2.66 [P<0.001]), which indicates patients who took proxalutamide had a 128% higher recovery rate than those treated with placebo (95% confidence interval 95 to 166%). No interaction effect of the primary outcome and gender was observed. See FIG. 8 (TABLE 3) and FIG. 7B.

Secondary outcomes. At the 28-day timepoint, a lower score distribution was observed for the proxalutamide group than in the placebo group. See FIG. 7A. The median ordinal scale score was 1 (interquartile range 1-1) for proxalutamide versus 7 (interquartile range 2 to 8) for placebo. No difference was observed between sexes upon stratification for both placebo and proxalutamide at twenty-eight days. See FIG. 7B. The overall 28-day recovery ratio was 1.81 (95% confidence interval 1.60 to 2.00), which indicates that patients who took proxalutamide had an 81% higher recovery rate than those treated with placebo (95% confidence interval 60 to 100). See FIG. 8 (TABLE 3).

A high risk of all-cause mortality was observed for placebo (49.4%; 95% confidence interval 44.0 to 54.7) compared to proxalutamide (11.0%; 95% confidence interval 8.0 to 14.9). The number needed to treat (NNT) to prevent one death from COVID-19 in hospitalized patients over twenty-eight days was 3 (95% confidence interval 3 to 2). The risk ratio for death was 0.22 (95% confidence interval 0.16 to 0.31), which indicates that treatment with proxalutamide reduced all-cause mortality rate over 28 days by 77.7%. Subgroup analysis by gender showed no interaction effects in the secondary outcome measures, See FIG. 8 (TABLE 3). Other subgroup analyses of the 28-day recovery ratio and all-cause mortality risk by city of the study site were performed.

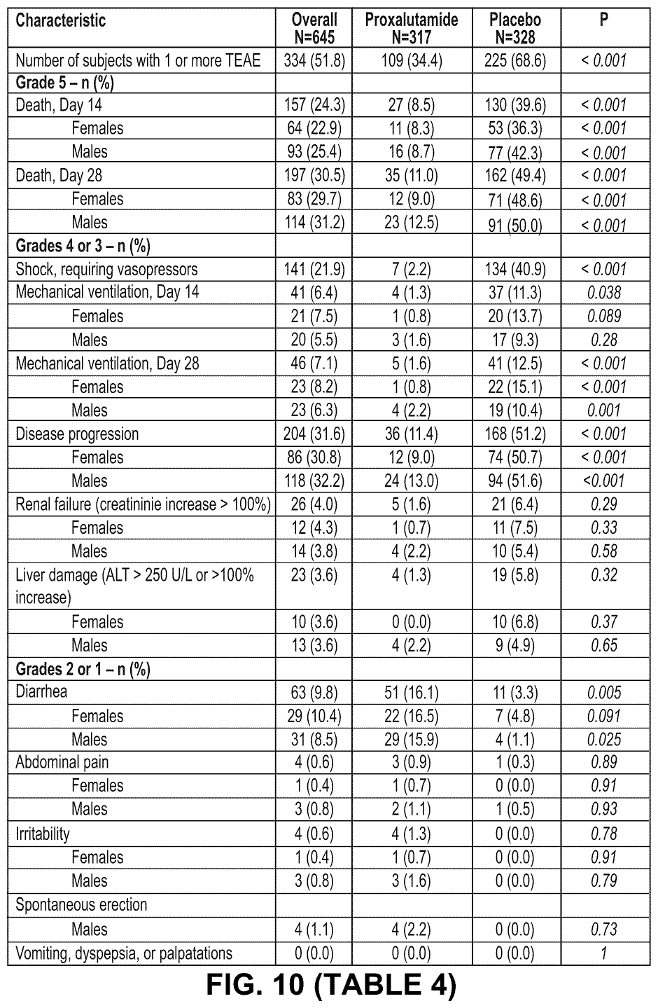

Safety outcomes. Adverse events observed during the trial are detailed in FIG. 10 (TABLE 4). Adverse events grades 4 or 3 were more commonly observed in the placebo arm (40.9%) than in the proxalutamide arm (2.2%), P<0.001. Diarrhea was the only adverse event reported at higher proportions in those receiving proxalutamide (16.1%) versus those receiving placebo (3.3%), P=0.005. Irritability was reported in four patients treated with proxalutamide (1.3%) and none in the placebo group. Among males taking proxalutamide, four reported spontaneous erection (2.2%) versus none in males taking placebo.

Introduction. SARS-COV-2 infects type II pneumocytes in the human lung and endothelial cells by anchoring to angiotensin-converting enzyme 2 (ACE2) receptors. Before binding to ACE2, spike proteins on the viral surface undergo structural modification via endogenous transmembrane protease, serine 2 (TMPRSS2). Hoffmann et al., Cell, 181(2), 271-280.e8 (2020). Hoffman proposed that inhibitors of TMPRSS2 would limit SARS-COV-2 infection. Hoffmann et al., Cell, 181(2), 271-280.e8 (2020). The TMPRSS2 promoter includes a 15 base pair androgen response element. Lin et al., Cancer Res. 59(17), 4180-4184 (1999). Thus, the inventors investigated anti-androgen drugs would be good candidates for limiting SARS-COV-2 entry into cells. Wambier et al., Drug Dev. Res. 81(7), 771-776 (2020). Pre-clinical studies showed that non-steroidal antiandrogens down regulate TMPRSS2 and inhibit viral replication in human cell culture. See Wu et al., SSRN Electron. J. (April 2020); Qiao et al., Proc. Natl. Acad. Sci. U.S.A., 118(1) (2020); Li et al., Nature Commun., 12(1), 866 (2021).

The inventors studied the use of proxalutamide in SARS-COV-2 positive men in an outpatient setting. In a double-blinded, placebo-controlled, randomized clinical trial, men treated with proxalutamide (200 mg/day) demonstrated reduced hospitalization rates, accelerated improvements of COVID-19 symptoms, and accelerated viral clearance. Applied Biology I. NCT04446429 Anti-Androgen Treatment for COVID-19. Clinicaltrials.gov. Published 2021. Proxalutamide also reduced the duration of COVID-19 in both men and women diagnosed with COVID-19 in the outpatient setting. Cadegiani et al., Cureus (February 2021). This EXAMPLE evaluated the efficacy of proxalutamide compared to the usual care in hospitalized men and women with COVID-19.

Trial design, setting and locations. This was a double-blinded, randomized, placebo-controlled, prospective, two-arm trial. The trial was conducted at eight centers in six cities of the state of Amazonas, Brazil from February 1 to Apr. 15, 2021, including enrollment and follow-up.

Inclusion criteria. Men and women hospitalized due to COVID-19 with a confirmed positive test for SARS-COV-2 within seven days before randomization. SARS-CoV-2 status was determined by real-time reverse transcription polymerase chain reaction (rtPCR) testing following the Cobas SARS-COV-2 rtPCR kit test protocol (Roche, USA). Subjects enrolled in this EXAMPLE were required to meet the following key acceptance criteria:
Admitted to the hospital with symptoms of COVID-19.
Male and females age ≥18 years old.
Laboratory confirmed positive SARS-COV-2 rtPCR test within seven days before randomization.
Clinical status on the COVID-19 Ordinal Scale of 3, 4, 5, or 6.
Coagulation: INR≤1.5×ULN, and APTT≤1.5×ULN.
Subject or legally authorized representative gives written informed consent before performing any EXAMPLE procedures.
Subject or legally authorized representative agree that subject will not participate in another COVID-19 trial while participating in this EXAMPLE.

Exclusion criteria included mechanical ventilation at the time of randomization, a history of congestive heart failure class III or IV (New York Heart Association), immunosuppression, alanine transferase (ALT) above five times ULN (>250 U/L), creatinine above 2.5 mg/ml, or a calculated eGFR was below 30 ml/min. Patients using any antiandrogen medications were also excluded. In female patients, those that were pregnant, breastfeeding, or were planning to become pregnant within ninety days were also excluded. Subjects were not to be enrolled if it was determined upon pre-study examination, they met these key criteria:
Subject enrolled in a study to investigate a treatment for COVID-19
Requires mechanical ventilation
Subject taking an anti-androgen of any type including androgen depravation therapy, 5-alpha reductase inhibitors, etc.
Patients allergic to the investigational product or similar drugs (or any excipients).
Subjects with malignant tumors in the past five years, except for completed resected basal cell and squamous cell skin cancer and completely resected carcinoma in situ of any type.
Subjects with known serious cardiovascular diseases, congenital long QT syndrome, torsade de pointes, myocardial infarction in the past 6 months, or arterial thrombosis, or unstable angina pectoris, or congestive heart failure classified as New York Heart Association (NYHA) class 3 or higher or left ventricular ejection fraction (LVEF)<50%, QTcF >450 milliseconds.
Subjects with uncontrolled medical conditions that could compromise participation in the study (e.g., uncontrolled hypertension, hypothyroidism, diabetes mellitus).
Known diagnosis of human immunodeficiency virus (HIV), hepatitis C, active hepatitis B, Treponema pallidum (testing is not mandatory)
Alanine Transaminase (ALT) or Aspartate Transaminase (AST)>5 times the upper limit of normal.
Estimated glomerular filtration rate (eGFR)<30 ml/minute.
Severe kidney disease requiring dialysis.
Women of child-bearing potential, defined as all women physiologically capable of becoming pregnant, unless they are using highly effective contraception, throughout the study and for 3 months after stopping GT0918 treatment. Highly effective contraception methods include the standard medical art-recognized methods.
Sexually active males must use a condom during intercourse while taking the drug and for 3 months after stopping treatment and should not father a child in this period. A condom must be used also by vasectomized men to prevent delivery of the drug via seminal fluid.
Subject likely to transfer to another hospital within the next twenty-eight days.
Subject (or legally authorized representative) not willing or unable to provide informed consent.

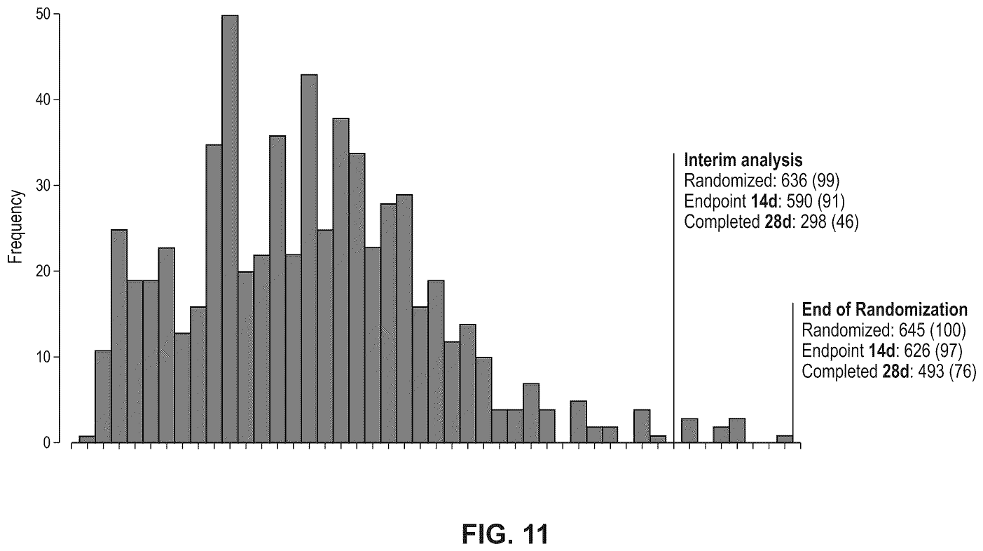

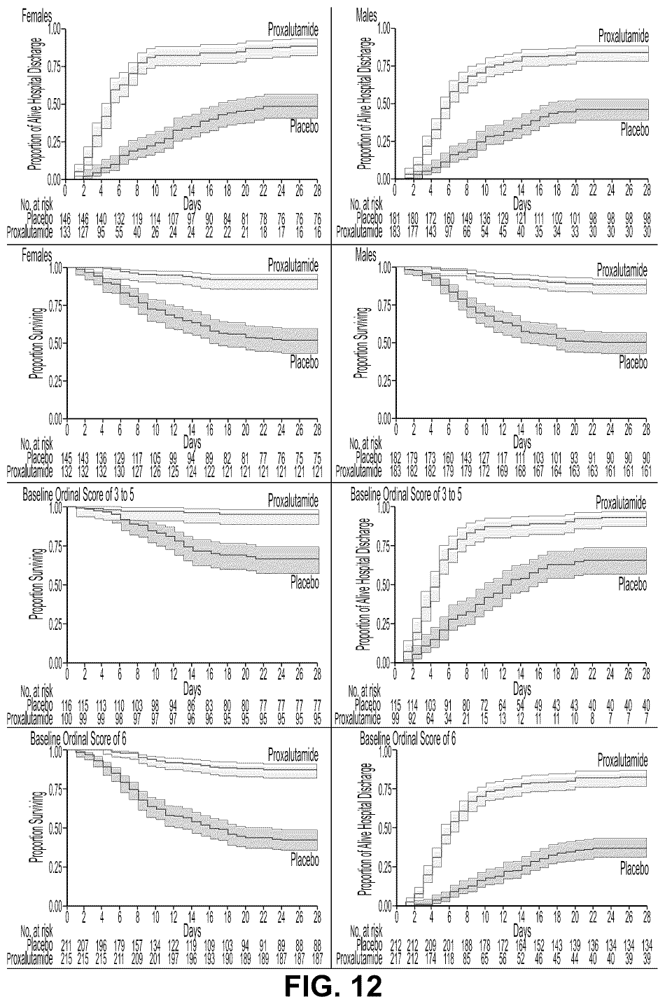

Patients were randomized to receive either proxalutamide 300 mg/day plus usual care or a placebo plus usual care for 14 days in a 1:1 ratio. If patients were discharged before 14 days, they were instructed to continue treatment. Therapy compliance was monitored daily.
Before the onset of the trial, a randomization table was created using a web-based randomization software (sealedenvelope.com/simple-randomiser/v1/lists) using 4, 6, and 8 block sizes and a list length for 662 treatment packages of identical appearance of either active or placebo group. A pharmacist sealed and labeled each package with a 3-character random-generated code. The packages contained six blister packs of seven individually sealed tablets, which accounted for forty-two tablets per treatment. The package instructions stated: Take three tablets by mouth once a day for fourteen consecutive days. The study was double-blinded with the identification of the group assignment known by the study monitor and the pharmacist who labeled the packages, who did not participate in dispensing the packages. Patients discharged before treatment day 14 had the remaining tablets dispensed as to complete the full 14-day treatment course and were actively evaluated for compliance daily until day 14. All centers followed the same protocol.
At the three hospital sites in Manaus, the treatments were dispensed by the hospital pharmacy at random. A procedure ensured non-sharing within the same site: boxes containing 5-50 treatment packages of the same blinded randomized treatment group were sealed for delivery to remote sites. Each box was delivered by a blinded research assistant directly to the research team at each remote site according to the local demand and enrollment capabilities. A new randomized box was delivered to the remote sites only after dispensing the previous box. The remote sites were not informed that each box contained a single arm and were instructed to follow the protocol and register in the case report forms the individual package 3-character code dispensed for each patient. This operation procedure introduced a bias in distributing drug and placebo between sites. FIG. 11 shows the enrollment over the time of the study. More proxalutamide was dispensed to the remote hospitals with fewer resources.

Procedures. The COVID-19 8-point ordinal scale was used for screening (day 0) and daily clinical assessments of patients on days 1-14 (or inpatient after day 14), and day 21, and day 28 (if outpatient), resulting in a maximum of seventeen data points for each patient. See Marshall et al., Lancet Infect. Dis., 20(8), e192-e197 (2020).

Participants discharged from the hospital were evaluated by investigators in follow-up appointments whenever possible, or by daily phone calls. Upon hospital discharge, patients were instructed to contact the same hospital and the local study team in case of relapse or new symptoms. Hospital readmissions were actively surveilled in all sites.

Baseline characteristics, previous medical history, and concomitant medications were recorded for each patient. Proxalutamide 300 mg/day or placebo plus usual care was given for fourteen days, even when COVID-19 remission occurred before this period. Usual care included medications such as enoxaparin, colchicine, methylprednisolone, dexamethasone, or antibiotic therapy if necessary.

Before the onset of the trial, a random sequence was created using a web-based randomization software using 4, 6, and 8 block sizes and a list length for 662 treatments. See Sealed Envelope Ltd. Create a blocked randomisation list. Simple Randomiser. Published 2021. The randomization sequence and the allocation concealment were performed remotely from the patient recruiting centers and it was not stratified by site. Pre-packing of tablets of either active or placebo group was manufactured to appear identical (Kintor Pharmaceuticals Ltd. Suzhou, China).

The local investigators who were directly involved with patient care, other healthcare providers, and patients were kept blinded to the group assignments until all patients completed the 28-day post-randomization period and the data was locked.

Outcomes and statistical analysis. The primary outcome measure was the 8-point COVD-19 ordinal scale at post-randomization day 14. The primary efficacy endpoint measure was the overall recovery ratio, which was calculated from recovery rates in each group. Recovery was defined as achieving alive hospital discharge (scores 1 and 2). The secondary outcome measures included recovery rate and all-cause mortality rate (score 8) and respective risk ratios at post-randomization day 28; all-cause mortality hazard ratio; median hospitalization time; and median post-randomization time to recover (alive hospital discharge). Subgroup analysis included sex and baseline scores.

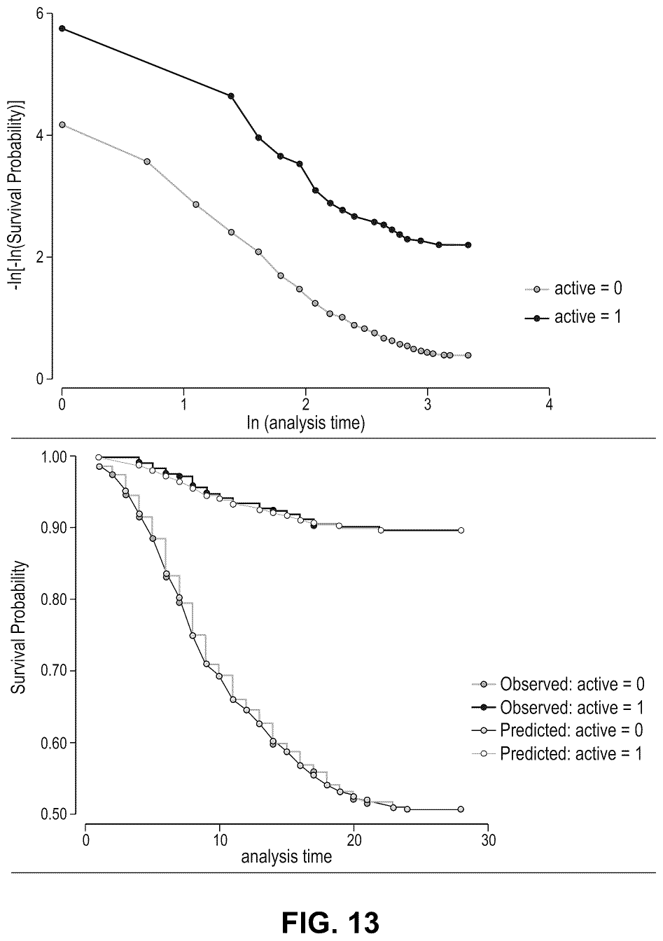

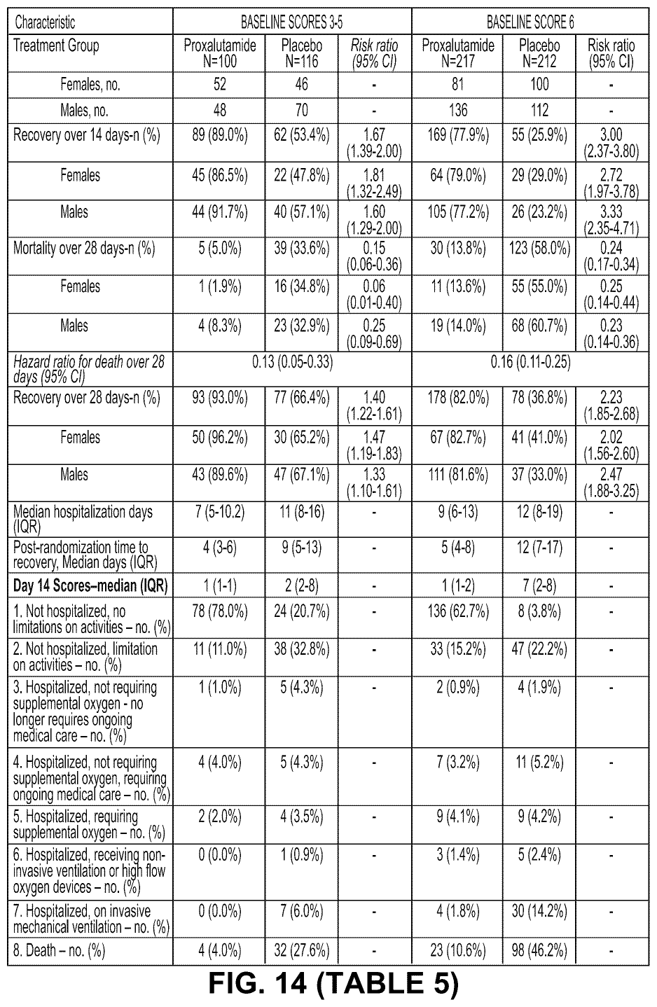

An intention-to-treat protocol was used for data analysis. The Wilcoxon Rank Sum test was used to assess the differences of the ordinal scale scores at 14 and 28 days. Risk ratios were calculated to measure the effects of proxalutamide versus placebo on the recovery and all-cause mortality rates. Additional analysis included the recovery and mortality risk ratios by gender, baseline COVID-19 ordinal score, and hospital site. To evaluate the all-cause mortality and recovery over the 28-days post-randomization observation period, Kaplan-Meier's survivor function estimated the proportion surviving and failure function for estimates of alive hospital discharges. Cox proportional hazards model was used to calculate hazard ratio (HR) for all-cause mortality over twenty-eight days and its 95% confidence interval (CI). Graphical assessment and Kaplan-Meier versus predicted survival showed that of the proportional-hazards assumption has not been violated. See FIG. 13.

The sample size was calculated to be able to detect a difference of approximately 14% in the overall recovery rate at fourteen days (risk ratio of 1.36) with a power of 90% and a type I error of 5%, over an estimated 39% recovery rate for the placebo group (based on the protocol for NCT04280705 [scenario 4]), and 3.5% non-compliance/cross over for each group. Statistical significance was set at P<0.05 and no correction for multiple comparisons was performed. Stata/SE version 16.1 for Mac (StataCorp LLC, College Station, TX, USA) was used to perform all statistical analysis.

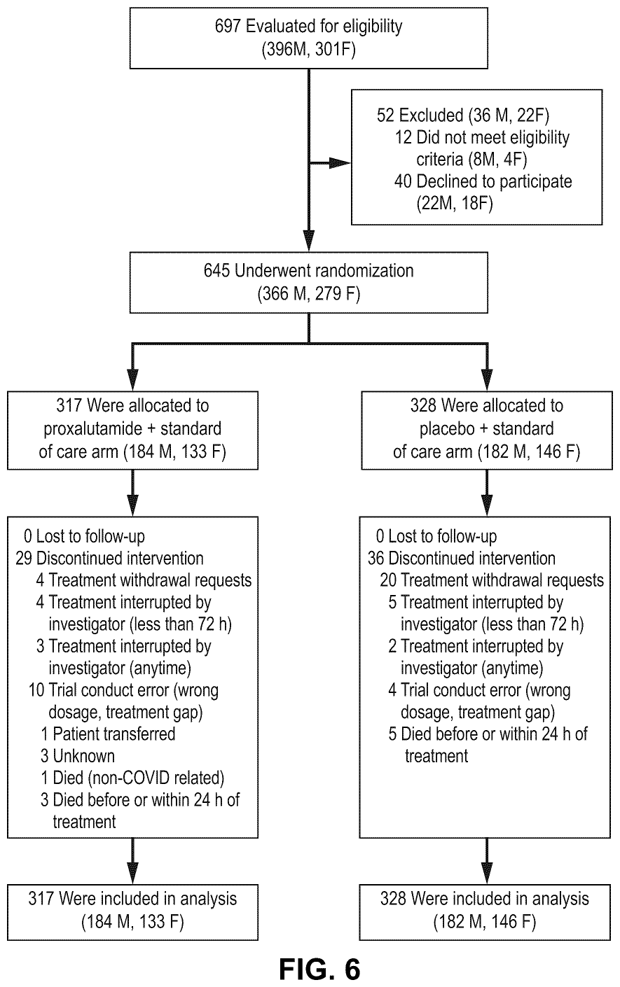

Patients. Between Feb. 1, 2021, and Mar. 17, 2021, 697 patients were assessed for eligibility, including 396 males (56.8%) and 301 females (43.2%). Twelve patients did not meet the eligibility criteria as they had kidney failure or liver abnormal enzymes (1.7%) and forty patients declined enrollment (5.7%). Of the 697 patients initially assessed, 645 underwent randomization. Three hundred seventeen patients were randomized to receive proxalutamide in addition to usual care, including 184 males (58.0%) and 133 females (42.0%). Three hundred twenty-eight were assigned to receive placebo in addition to usual care, including 182 males (55.5%) and 146 females (44.5%). After randomization, 29 of the 317 (9.1%) patients receiving proxalutamide and 36 of the 328 (10.9%) receiving placebo did not complete the trial protocol, the reasons are described in FIG. 6.

Discussion This EXAMPLE demonstrates in a randomized, double-blind, placebo-controlled clinical trial that the use of proxalutamide, a second-generation nonsteroidal antiandrogen, reduced mortality, length of stay, and significantly improved clinical outcomes of hospitalized COVID-19 patients. These findings are consistent with previous reports of the efficacy of proxalutamide in an outpatient setting. See, Cadegiani et al., Cureus (February 2021); Marshall et al., Lancet Infect. Dis., 20(8), e192-e197 (2020); McCoy et al., Front. Med. (2021). These findings are also consistent with reports using other antiandrogen regimens, including dutasteride and finasteride. Cadegiani et al., Cureus, (February 2021); Zarehoseinzade et al., MJIRI, 35(1), 232-237 (2021).

Proxalutamide was a benefit to COVID-19 patients who were severely ill. At the time of the trial, the state of Amazonas experienced a surge in COVID-19 cases resulting in overcrowding at hospitals. As such, prioritized admission to hospitals resulted in admission of the most severely affected patients with COVID-19. The mortality rate was exceptionally high (49.4%) in the placebo group. Though the observed mortality rate may have been influenced by the emergent P.1 strain, the high mortality rate in the placebo group was not unexpected based on past reports. See Ranzani et al., Lancet Respir Med. (2021). The northern Amazonas region of Brazil reported high mortality rates, ranging from 24-55% amongst hospitalized COVID-19 patients as early as April of 2020, while the average rates observed in all of Brazil range from 14-38%. Brasil. Ministério da Saúde. Agência Nacional de Vigilância Sanitária. SRAG 2021—Severe Acute Respiratory Syndrome Database—including data from COVID-19—SRAG Apr. 12, 2021.

As of March 2021, the P.1 lineage was responsible for at least 70% of the current SARS-COV-2 genomes sequenced in Brazil, and for at least 90% of the SARS-CoV-2 genomes in the state of Amazonas. See Franceschi et al., medRxiv, 2021.03.08.21253152 (January 2021). In a post-hoc analysis, sequencing of viral genomes from patient samples obtained from the study sites and during the trial dates were found to be the P.1 lineage in all but one case. P.1 is one of most relevant variants of concern (VOC) worldwide. This variant demonstrated enhanced affinity for ACE-2 binding, potentially resulting in higher viral loads. Golubchik et al., medRxiv, 2021.01.12.20249080 (January 2021). The E484K substitution, previously demonstrated by the inventors as being positively selected in diverse lineages in Brazil, can induce immune escape with consequent reinfection and failure of other therapeutic modalities, such as convalescent plasma. Ferrareze et al., bioRxiv, 2021.01.27.426895 (January 2021); Nelson et al., bioRxiv. 2021.01.13.426558 (January 2021). Proxalutamide was effective in a region with high P.1 prevalence.

The mortality rate of patients treated with remdesivir was 11.4% at 29 days compared to 15.2% in the placebo group, though not reaching statistical significance (Hazard Ratio 0.73; 95% CI, 0.52 to 1.03). Beigel et al., N. Engl. J. Med., 383(19): 1813-1826 (2020). Similarly, the combination of remdesivir and baricitinib trended to a reduction in 28-day mortality from 7.8% to 5.1% (Hazard Ratio 0.65; 95% confidence interval, 0.39 to 1.09). The clinical benefits of proxalutamide were superior to either of these treatments with greater than 77.7% reduction in 28-day mortality rate under intention-to-treat (ITT) analysis (Hazard Ratio of 0.16; 95% confidence interval, 0.11 to 0.24). Dexamethasone benefits the most severe COVID-19 patients. Among those patients requiring mechanical ventilation, dexamethasone was shown to reduce mortality compared to placebo (29.3% vs. 41.4%; rate ratio, 0.64; 95% confidence interval, 0.51 to 0.81). All of the participants in this trial received corticosteroids, but there was still a survival advantage to receiving proxalutamide.

Treatment emergent adverse events associated with proxalutamide were limited to diarrhea. Diarrhea was more frequent among the proxalutamide group in an outpatient trial. Cadegiani et al., Cureus (February 2021). Severe adverse events of renal failure and hepatic damage were associated with placebo. See FIG. 10 (TABLE 4). This is a natural progression of COVID-19 in the placebo group, which further supports consideration of proxalutamide as a therapy for hospitalized patients. While Phase 1 safety data for proxalutamide supports a use for twenty-eight days in both men and women, proxalutamide was studied primarily in prostate cancer patients, therefore, long term side effects should be assessed in future studies. Other approved molecules of the same class may show similar results, such as apalutamide, enzalutamide, darolutamide, bicalutamide, or flutamide.

Limitations. First, the remote locations of many of the study sites created operational difficulties that led to an unbalanced distribution of proxalutamide and placebo amongst sites. The possibility of effect sizes skewing in favor of the proxalutamide group due to its disproportional concentration in the smaller hospitals is unlikely because a similar size effect was observed in subgroup analysis of all hospital sites. Second, the severity of patients admitted to Amazonas hospitals during the trial did not allow testing proxalutamide in many hospitalized patients who did not require supplemental oxygen (ordinal score 3). However, the inventors showed that proxalutamide reduced hospitalizations and improved symptom recovery and viral clearance in outpatients with mild-moderate disease. Applied Biology I. NCT04446429 Anti-Androgen Treatment for COVID-19. Clinicaltrials.gov. Published 2021. The subgroup analysis showed patients with baseline scores of 3-5 benefited from proxalutamide. Third, more patients used colchicine in the placebo arm despite randomization, which most likely reflects additional therapeutic interventions associated with longer hospitalization in the placebo group. Colchicine is unlikely to have contributed to increased mortality directly as there is either no effect, with 28-day mortality rate ratio of 1.01 in the RECOVERY platform trial (95% CI, 0.93 to 1.10) or limited benefit in COVID-19. See Horby et al., medRxiv, 2021.05. 18.21257267 (January 2021); Manenti et al., PLOS One, 16(3), e0248276 (2021); Hariyanto et al., Clin. Exp. Pharmacol. Physiol. (March 2021); Salah et al., Am. J. Cardiol., 145, 170-172 (2021). Fourth, remdesivir was not available to the patients. But patients receiving proxalutamide had a substantial benefit. Lastly, a dropout rate of approximately 9-11% was observed in both study arms. The explanation for this participant attrition varied and was seen in both arms. Proxalutamide retained its efficacy in the ITT analysis.

Women treated with proxalutamide responded similarly to proxalutamide-treated men, See FIG. 10 (TABLE 4) and FIG. 7. The significant improvement observed in severely-ill patients appeared disproportionate to the proposed mechanism of reducing viral entry (FIG. 10 (TABLE 4), Baseline score 6).

SARS-COV-2 lineage determination. For a post hoc analysis, clinical samples from patients admitted to one of the participating centers testing positive for SARS-COV-2 in a first RT-qPCR had their samples submitted to a second RT-qPCR performed by BiomeHub (Florianópolis, Santa Catarina, Brazil), using charite-berlin protocol. Only samples with quantification cycle (Cq) below 30 for at least one primer were processed for SARS-COV-2 genome sequencing by the BiomeHub laboratory. To perform the SARS-COV-2 genome sequencing, total RNAs were prepared as in the reference protocol using SuperScript IV (Invitrogen) for cDNA synthesis and Platinum Taq High Fidelity (Invitrogen) for specific viral amplicons. The cDNA obtained were subsequently used for the library preparation with Nextera Flex (Illumina) and quantified with Picogreen and Collibri Library Quantification Kit (Invitrogen). The sequencing was performed on MiSeq 150×150 runs with 500×SARS-COV-2 coverage (50-100 mil reads/per sample). The SARS-COV-2 genome assembly was generated by an in-house pipeline from BiomeHub (Florianópolis, Santa Catarina, Brazil). The remotion of adapters and read trimming in 150 nucleotides were performed with fastqtools.py, followed by the reads mapping to the reference SARS-COV-2 genome (GenBank accession number NC_045512.2) with Bowtie v2.4.2 (additional parameters: end-to-end and very-sensitive). The mapping coverage and sequencing depth were obtained with samtools v1.11 (minimum base quality per base (Q) ≥ 30). Consensus genome sequences were then generated with bcftools mpileup (Q ≥ 30; depth (d)≤ 1,000) combined with bcftools filter (DP>50) and bcftools consensus v1.11. Finally, the identification of the SARS-COV-2 virus lineages was performed by the Pangolin v2.3.8 web server.

### LIST OF EMBODIMENTS

Specific compositions and methods of androgen receptor inhibition to treat sepsis and shock have been described. The scope of the invention should be defined solely by the claims. A person having ordinary skill in the art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as those skilled in the art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

### REFERENCES

A person having ordinary skill in the medical art can use these scientific references as guidance to predictable results when making and using the invention.
Applied Biology I. NCT04446429 Anti-androgen treatment for COVID-19. Clinicaltrials.gov. Published 2021.
Beigel et al., Remdesivir for the Treatment of Covid-19—Final Report. N. Engl. J. Med., 383(19): 1813-1826 (2020).
Brasil, Anvisa aprova registro da vacina da Fiocruz/AstraZeneca e de medicamento contra o coronavírus—O imunizante e o antiviral Rendesivir podem ser usados amplamente no país. gov.br. Published 2021.
Brasil. Ministério da Saúde. Agência Nacional de Vigilância Sanitária. SRAG 2021—Severe Acute Respiratory Syndrome Database—including data from COVID-19—SRAG Apr. 12, 2021
Cadegiani et al., Clinical symptoms of hyperandrogenic women diagnosed with COVID-19. J. Eur. Acad. Dermatol. Venereol., 35(2) (2021).
Cadegiani et al., Clinical symptoms of hyperandrogenic women diagnosed with COVID-19. J. Eur. Acad. Dermatology Venereol., jdv. 17004 (November 2020).
Cadegiani et al., Proxalutamide significantly accelerates viral clearance and reduces time to clinical remission in patients with mild to moderate COVID-19: Results from a randomized, double-blinded, placebo-controlled trial. Cureus. February 2021.
Cadegiani, Lin, Goren, & Wambier, Potential risk for developing severe COVID-19 disease among anabolic steroid users. BMJ Case Rep., 14(2), e241572 (2021).
Cadegiani, McCoy, Gustavo Wambier, & Goren. Early anti-androgen therapy with dutasteride reduces viral shedding, inflammatory responses, and time-to-remission in males with COVID-19: A Randomized, double-blind, placebo-controlled interventional trial (EAT-DUTA AndroCoV Trial—Biochemical). Cureus (February 2021).
Cadegiani, Pre-RCT AndroCoV Trial—COVID-19 public dataset 2020-10-31. OSF. Published 2020.
Clinckemalie et al., Androgen regulation of the TMPRSS2 gene and the effect of a SNP in an androgen response element. Molecular Endocrinology, Volume 27, Issue 12, pages 2028-2040 (Dec. 1, 2013).
Dexamethasone in Hospitalized Patients with Covid-19—Preliminary Report. N. Engl. J. Med., 384(8) (2020).
Dhurat et al., Spironolactone in adolescent acne vulgaris. Dermatol. Ther., 34(1) (2021).
Elalfy et al., Effect of a combination of nitazoxanide, ribavirin, and ivermectin plus zinc supplement (MANS.NRIZ study) on the clearance of mild COVID-19. J. Med. Virol., 93(5), 3176-3183 (2021).
Ferrareze et al., E484K as an innovative phylogenetic event for viral evolution: Genomic analysis of the E484K spike mutation in SARS-COV-2 lineages from Brazil. bioRxiv, 2021.01.27.426895 (January 2021).
Franceschi et al., Mutation hotspots, geographical and temporal distribution of SARS-COV-2 lineages in Brazil, February 2020 to February 2021: insights and limitations from uneven sequencing efforts. medRxiv, 2021.03.08.21253152 (January 2021).
Golubchik et al., Early analysis of a potential link between viral load and the N501Y mutation in the SARS-COV-2 spike protein. medRxiv, 2021.01.12.20249080 (January 2021).
Goren et al., A preliminary observation: Male pattern hair loss among hospitalized COVID-19 patients in Spain—A potential clue to the role of androgens in COVID-19 severity. J. Cosmet. Dermatol., 19(7): 1545-1547 (July 2020).
Goren et al., Anti-androgens may protect against severe COVID-19 outcomes: results from a prospective cohort study of 77 hospitalized men. J. Eur. Acad. Dermatol. Venereol. (2020).
Goren et al., What does androgenetic alopecia have to do with COVID-19? An insight into a potential new therapy. Dermatol Ther., 33(4), e13365 (July 2020).
Hariyanto et al., Colchicine treatment can improve outcomes of coronavirus disease 2019 (COVID-19): A systematic review and meta-analysis. Clin. Exp. Pharmacol. Physiol. (March 2021).
Hoffmann et al., SARS-COV-2 cell entry depends on ACE2 and TMPRSS2 and is blocked by a clinically proven protease inhibitor. Cell, 181(2), 271-280.e8 (2020).
Horby et al., Colchicine in patients admitted to hospital with COVID-19 (RECOVERY): a randomised, controlled, open-label, platform trial. medRxiv, 2021.05. 18.21257267 (January 2021)
Johnston et al., Hydroxychloroquine with or without azithromycin for treatment of early SARS-COV-2 infection among high-risk outpatient adults: A randomized clinical trial. EClinicalMedicine, 33, 100773 (2021).
Kalil et al., Baricitinib plus remdesivir for hospitalized adults with Covid-19. N. Engl. J. Med., 384(9), 795-807 (2021).
Kanji et al., False negative rate of COVID-19 PCR testing: a discordant testing analysis. Virol. J., 18(1), 13 (2021).
Lee, Yousaf, Fang, & Kolodney. Male balding is a major risk factor for severe COVID-19. J. Am. Acad. Dermatol. (July 2020).
Letko et al., Functional assessment of cell entry and receptor usage for SARS-COV-2 and other lineage B betacoronaviruses. Nature Microbiology, Volume 5, pages 562-569 (Feb. 24, 2020).
Li et al., Distinct mechanisms for TMPRSS2 expression explain organ-specific inhibition of SARS-COV-2 infection by enzalutamide. Nature Commun., 12(1), 866 (2021).
Lin et al., Prostate-localized and androgen-regulated expression of the membrane-bound serine protease TMPRSS2. Cancer Res., 59(17), 4180-4184 (1999).
López-Medina et al., Effect of Ivermectin on Time to Resolution of Symptoms Among Adults with Mild COVID-19: A Randomized Clinical Trial. JAMA, 325(14), 1426-1435 (2021).
Manenti et al., Reduced mortality in COVID-19 patients treated with colchicine: Results from a retrospective, observational study. PLOS One, 16(3), e0248276 (2021).
Marshall et al., A minimal common outcome measure set for COVID-19 clinical research. Lancet Infect. Dis., 20(8), e192-e197 (2020).
McCoy et al., 5-alpha-reductase inhibitors are associated with reduced frequency of COVID-19 symptoms in males with androgenetic alopecia. J. Eur. Acad. Dermatol. Venereol., 35(4), e243-e246 (April 2021).
McCoy et al., 5-alpha-reductase inhibitors are associated with reduced frequency of COVID-19 symptoms in males with androgenetic alopecia. J. Eur. Acad. Dermatol. Venereol., jdv. 17021 (November 2020).
McCoy et al., Androgen receptor genetic variant predicts COVID-19 disease severity: a prospective longitudinal study of hospitalized COVID-19 male patients. J. Eur. Acad. Dermatol. Venereol., 35(1), e15-e17 (January 2021).
McCoy et al., Proxalutamide (GT0918) reduces the rate of hospitalization for COVID-19 male outpatients: A randomized double-blinded placebo-controlled trial. Front. Med. (2021).
McCoy et al., Racial variations in COVID-19 deaths may be due to androgen receptor genetic variants associated with prostate cancer and androgenetic alopecia. Are anti-androgens a potential treatment for COVID-19? J. Cosmet. Dermatol., 19(7), 1542-1543 (July 2020).
Meng, Deng, Dai, & Meng, COVID-19 and anosmia: A review based on up-to-date knowledge. Am. J. Otolaryngol., 41(5), 102581 (2020).
Montopoli et al., Androgen-deprivation therapies for prostate cancer and risk of infection by SARS-COV-2: a population-based study (N=4532). Ann Oncol. 2020 August;31(8): 1040-1045.
Nelson et al., Molecular dynamic simulation reveals E484K mutation enhances spike RBD-ACE2 affinity and the combination of E484K, K417N and N501Y mutations (501Y.V2 variant) induces conformational change greater than N501Y mutant alone, potentially resulting in an escap. bioRxiv, 2021.01.13.426558 (January 2021).
Nuffield Department of Population Health. RECOVERY trial closes recruitment to colchicine treatment for patients hospitalised with COVID-19. Published 2021.
Qiao et al., Targeting transcriptional regulation of SARS-COV-2 entry factors ACE2 and TMPRSS2. Proc. Natl. Acad. Sci. U.S.A. 118(1) (2020).
Qu et al., Metabolomic profiling to evaluate the efficacy of proxalutamide, a novel androgen receptor antagonist, in prostate cancer cells. Invest New Drugs. 2020; 38(5): 1292-1302.
Ranzani et al., Characterisation of the first 250 000 hospital admissions for COVID-19 in Brazil: A retrospective analysis of nationwide data. Lancet Respir. Med. (2021).
Rocco et al., Early use of nitazoxanide in mild Covid-19 disease: Randomised, placebo-controlled trial. Eur Respir J. (January 2021).
Rossignol et al., Early treatment with nitazoxanide prevents worsening of mild and moderate COVID-19 and subsequent hospitalization. medRxiv, 2021.04.19.21255441 (January 2021).
Salah & Mehta, Meta-analysis of the effect of colchicine on mortality and mechanical ventilation in COVID-19. Am. J. Cardiol., 145, 170-172 (2021).
Salazar Arenas et al., Alopecia and severity of COVID-19: A cross-sectional study in Peru. Le Infez. Med., 29(1), 37-45 (2021).
Samuel et al., Androgen signaling regulates SARS-COV-2 receptor levels and is associated with Severe COVID-19 symptoms in men. Cell Stem Cell, 27(6), 876-889.e12 (2020).
Sealed Envelope Ltd. Create a blocked randomisation list. Simple Randomiser. Published 2021.
Subramanian et al., Increased COVID-19 infections in women with polycystic ovary syndrome: A population-based study. Eur. J. Endocrinol., 184(5), 637-645 (2021).
The results of Brazil trial for use in COVID-19 (released on YouTube by Samel Hospital in Brazil, Mar. 10, 2021.
Tran et al., Development of a second-generation antiandrogen for treatment of advanced prostate cancer. Science, 324(5928), 787-790 (2009).
United States Food & Drug Administration. Avodart® (dutasteride) Soft Gelatin Capsules. Highlights of Prescribing Information. Published 2008.
Update to living systematic review on drug treatments for Covid-19. BMJ, n858 (March 2021).
Vicenzi et al., The efficacy of the mineralcorticoid receptor antagonist canrenone in COVID-19 patients. J. Clin. Med., 9(9) (2020).
Wambier & Goren, Severe acute respiratory syndrome coronavirus 2 (SARS-COV-2) infection is likely to be androgen mediated. J. Am. Acad. Dermatol., 83(1), 308-309 (2020).
Wambier et al., Androgen sensitivity gateway to COVID-19 disease severity. Drug Dev Res. 2020; 81(7):771-776.
Wambier et al., Androgenetic alopecia in COVID-19: Compared to age-matched epidemiologic studies and hospital outcomes with or without the Gabrin sign. J. Am. Acad. Dermatol. (July 2020).
Wambier et al., Androgenetic alopecia present in the majority of patients hospitalized with COVID-19: The “Gabrin sign.” J. Am. Acad. Dermatol., 83(2), 680-682 (August 2020).
Wambier, Mehta, Goren, & Cadegiani, COVID-19, androgens, and androgenic alopecia. Dermatological Rev., der2.50. (2020).
Wambier, Pereira, Prado Júnior, & Foss, Brazilian blood donation eligibility criteria for dermatologic patients. An Bras Dermatol. 87(4), 590-595 (2012).
WHO Coronavirus (COVID-19) Dashboard.
Wu et al., Suppression of androgen receptor (AR)-ACE2/TMPRSS2 axis by AR antagonists may be therapeutically beneficial for male COVID-19 patients. SSRN Electron. J. (April 2020).
Zarehoseinzade et al., Finasteride in hospitalized adult males with COVID-19: A risk factor for severity of the disease or an adjunct treatment: A randomized controlled clinical trial. MJIRI, 35(1), 232-237 (2021).

We claim:

1. A method for applying diarylhydantoin compounds, including flutamide, bicalutamide, enzalutamide, apalutamide, and proxalutamide, to treat sepsis and conditions that mimic sepsis, including COVID-19 infection.

2. The method of claim 1, wherein the diarylhydantoin compound is proxalutamide.

3. A method for applying diarylhydantoin compounds, including flutamide, bicalutamide, enzalutamide, apalutamide, and proxalutamide, to treat traumatic injury.
